(19) United States
(12) Patent Application Publication
(10) Pub. No.: US 2015/0216593 A1
(43) Pub. Date: Aug. 6, 2015

(54) TRANSPEDICULAR ACCESS TO THE INTERVERTEBRAL DISC SPACE FOR DISCECTOMY, END PLATE PREPARATION, AND INTERBODY FUSION

(71) Applicant: The University of Toledo, Toledo, OH (US)

(72) Inventor: Ashok Biyani, Sylvania, OH (US)

(73) Assignee: The University of Toledo, Toledo, OH (US)

(21) Appl. No.: 14/686,119

(22) Filed: Apr. 14, 2015

Related U.S. Application Data
(62) Division of application No. 12/484,439, filed on Jun. 15, 2009, now abandoned.
(60) Provisional application No. 61/061,582, filed on Jun. 13, 2008.

(51) Int. Cl. A61B 18/14; A61B 17/16

(52) U.S. Cl. CPC A61B 18/1492; A61B 17/1671; A61B 17/1659; A61B 17/3403

(57) ABSTRACT

An insertion device is configured to access a disc positioned between adjacent vertebrae. The insertion device includes a cannula having a passage formed therein. The cannula has an exit aperture. An obturator is substantially positioned within the passage formed in the cannula. One end of the obturator has a probe and the other end of the obturator has a head. An impaction cap is in contact with the cannula and is positioned to cover the head of the obturator. The impaction cap is configured to allow at least a portion of the cannula to be inserted through a portion of one vertebra without deployment of the probe of the obturator through the exit aperture of the cannula.

### STATEMENTS REGARDING FEDERALLY SPONSORED RESEARCH OR DEVELOPMENT AND CROSS-REFERENCE TO RELATED APPLICATIONS

This invention was not made with any government support. This application is a divisional patent application of U.S. patent application Ser. No. 12/484,439, filed Jun. 15, 2009, which claims the benefit of U.S. Provisional Application No. 61/061,582 filed Jun. 13, 2008. The disclosures of both of these applications are incorporated herein by reference.

### BACKGROUND OF THE INVENTION

This invention relates to the field of orthopedic surgery and more particularly to the area of spinal surgery. One example of spinal surgery is a procedure called spinal fusion. Spinal fusion is a procedure that involves the joinder of two or more vertebrae. Spinal fusion is routinely performed for a wide variety of spinal disorders. Examples of spinal fusion techniques include posterolateral fusion and interbody fusion.

During an interbody spinal fusion procedure, a bone graft is positioned between the vertebrae in the area usually occupied by an intervertebral disc. In preparation for the spinal fusion, the intervertebral disc can be partially or fully removed. A device may be positioned in the space left by the removed intervertebral disc to maintain spine alignment and spacing between adjacent vertebrae.

Interbody fusion can be performed from several different entry directions, including but not limited to an anterior approach, a posterior approach, a transforaminal approach, or a direct lateral approach. Interbody fusion procedures also can involve minimally invasive fusion techniques resulting in minimal incisions, reduced recovery time, and shortened hospital stays. However, many minimally invasive fusion techniques have some drawbacks. As one example, a Transaxial Anterior Lumbar Interbody Fusion (“AXIALIF”) procedure is usually performed only at L5-S1 level. As another example, Extreme or Direct Lateral Interbody Fusion (“XLIF” or “DLIF”) approaches can have a high rate of thigh pain and typically cannot be performed at L5-S1 level. Also, minimally invasive Transforaminal Lumbar Interbody Fusion (“TLIF”) surgery involves facet resection. TLIF also typically involves a hemilaminectomy and mobilization of the nerve root, which can lead to neuropraxia and perineural scarring. TLIF may also require removal of a portion of the annulus fibrosus, which can lead to a risk of cage migration in the postoperative period. Thus, it would be desirable to provide an improved method of accessing the disc space for procedures including a discectomy, end plate preparation, and interbody fusion that overcomes aforementioned drawbacks.

### SUMMARY OF THE INVENTION

This invention relates to an insertion device that is configured to access a disc positioned between adjacent vertebrae. The insertion device includes a cannula having a passage formed therein. The cannula has an exit aperture. An obturator is positioned within the passage formed in the cannula. One end of the obturator has a probe and the other end of the obturator has a head. An impaction cap is in contact with the cannula and is positioned to cover the head of the obturator. The impaction cap is configured to allow at least a portion of the cannula to be inserted through a portion of one vertebra without deployment of the probe of the obturator through the exit aperture of the cannula.

According to this invention, there is also provided an insertion device configured to access a disc positioned between adjacent vertebrae. The insertion device includes a cannula having an angled passage formed therein. The cannula has a longitudinal axis and an exit aperture. At least a portion of the passage forms an angle with respect to the longitudinal axis. An obturator is substantially positioned within the angled passage formed in the cannula. One end of the obturator has a probe and the other end of the obturator has a head. The angle formed between the longitudinal axis of the cannula and the angled passage is in a range of from about 5° to about 60°.

According to this invention there is also provided a probe assembly configured for ablation of disc material. The probe assembly includes a plurality of probe filaments and an inflatable device positioned adjacent to a portion of the probe filaments. A cable is connected to the probe filaments and the inflatable device. The probe filaments are configured for ablation of disc material using RF radiofrequency waves.

According to this invention there is also provided an abrading assembly configured to abrade disc material. The abrading assembly includes an abrading head and an actuator connected to the abrading head. The actuator is configured to rotate the abrading head. The abrading head and the actuator are configured to be guided by a guide wire.

According to this invention there is also provided a portal cannula configured to extend from an opening in a vertebral element outwardly past an exterior surface of skin. The portal cannula includes a distal end configured to be positioned exterior to the skin. An elongated center portion is connected to the distal end. A proximal end has one end connected to elongated center portion and another end positioned over the opening in the vertebral element. The portal cannula is anchored in place by an inflatable device connected to the portal cannula and positioned beneath the exterior surface of the skin.

According to this invention there is also provided a pedicular anchor configured to fix medical implements to vertebral elements. The pedicular anchor includes a threaded portion configured for insertion into vertebral elements and a shank portion connected to the threaded portion. A head is pivotably connected to the shank portion, the head being configured to receive a medical implement. The shank portion has an outer surface compatible with minor violations of the vertebral element.

According to this invention there is also provided a method of providing transpedicular access to intervertebral disc space for medical procedures. The method includes the steps of providing an insertion device, the insertion device having a cannula with a passage formed therein, the cannula having an exit aperture, an obturator is substantially positioned within the passage formed in the cannula, one end of the obturator has a probe and the other end of the obturator has a head, an impaction cap is in contact with the cannula and is positioned to removably cover the head of the obturator, the impaction cap is configured to allow the insertion device to be inserted through a portion of one vertebra without deployment of the probe of the obturator through the exit aperture of the cannula, urging a distal end of the cannula into a vertebra by applying force to the impaction cap, removing the impaction cap to expose the passage in the cannula, urging the probe of the obturator through the exit aperture of the cannula such as to form a curved bone tunnel in the vertebra, removing the obturator from the curved bone tunnel and accessing the intervertebral disc space through the curved bone tunnel for the intended medical procedure.

According to this invention there is also provided a probe assembly configured for ablation of disc material from between opposing vertebrae. The probe assembly includes a probe element having a fixed diameter. A guide wire is connected to the probe element and configured to guide the probe element through a curved bone tunnel formed in one vertebra. A cable is connected to the probe element. The probe element is configured for ablation of disc material using RF radiofrequency waves.

According to this invention there is also provided an insertion device configured to access a disc positioned between adjacent vertebrae. The insertion device includes a cannula having a tip and an angled passage formed therein. A first end of the angled passage is located a distance of from about 30 mm to about 60 from the cannula tip and a second end of the angled passage aligns with the deflecting surface on the upper shank. The angled passage is configured to guide a guide wire from the first end to the second end. The guide wire is directed by the second end of the angled passage and the deflecting surface on the tip to the disc.

According to this invention there is also provided an insertion device configured to access a disc positioned between adjacent vertebrae. The insertion device includes a cannula having a passage. An obturator is substantially positioned in the passage of the cannula. The obturator has a head, a distal end and an angled passage formed therein. The distal end of the obturator has a deflecting surface. A first end of the angled passage is positioned in the head and a second end of the passage extends to the deflecting surface. The angled passage is configured to guide a guide wire from the first end to the second end. The guide wire is directed by the second end of the angled passage to the disc.

Various aspects of this invention will become apparent to those skilled in the art from the following detailed description of the preferred embodiment, when read in light of the accompanying drawings.

### BRIEF DESCRIPTION OF THE DRAWINGS

FIG. 1 is a cross-sectional view of a first embodiment of an insertion device illustrated in a pre-insertion configuration.

FIG. 2 is a cross-sectional view of the insertion device of FIG. 1 illustrated after insertion into a vertebra.

FIG. 3 is a cross-sectional view of the insertion device of FIGS. 1 and 2 illustrated with a deployed obturator.

FIG. 4 is a side view, partially in cross-section, of a portion of the insertion device of FIG. 3 illustrated with the deployed obturator.

FIG. 5a is a plan view of a disc illustrating the penetration of the deployed obturator of FIG. 4 into a first location of a nucleus pulposus.

FIG. 5b is a plan view of a disc illustrating the penetration of the deployed obturator of FIG. 4 into a second location of the nucleus pulposus.

FIG. 6 is an exploded cross-sectional view of a second embodiment of the cannula of FIG. 1 including an insertable deflector.

FIG. 7 is a cross-sectional view, partially broken away, of a third embodiment of an insertion device illustrated in a pre-insertion configuration.

FIG. 8 is a cross-sectional view, partially broken away, of the insertion device of FIG. 7 illustrated with a deployed obturator.

FIG. 9 is a cross-sectional view, partially broken away, of a fourth embodiment of an insertion device illustrated with a deployed obturator.

FIG. 10 is a cross-sectional view of fifth embodiment of an insertion device illustrated in a pre-insertion configuration.

FIG. 11 is a cross-sectional view of the insertion device of FIG. 10 illustrated with a deployed obturator.

FIG. 12 is a cross-sectional view of a portion of a sixth embodiment of an insertion device illustrating passage of the insertion device over a guide wire.

FIG. 13 is a cross-sectional view of a portion of a seventh embodiment of an insertion device illustrated with a deployed obturator.

FIG. 14 is a cross-sectional view of an eighth embodiment of an insertion device illustrated in a pre-insertion configuration.

FIG. 15 is a cross-sectional view of the insertion device of FIG. 14 illustrated with a deployed obturator.

FIG. 16 is a cross-sectional view of a portion of a ninth embodiment of an insertion device illustrating an obliquely placed passage way for insertion of a guide wire.

FIG. 17 is a cross-sectional view in elevation of a tenth embodiment of an insertion device illustrating an obliquely placed passage way for the insertion of the guide wire.

FIGS. 18a, 18b, 18c, 18d, and 18e are end elevational views in cross-section of five different embodiments of a cannula having an internal passage.

FIGS. 19a, 19b, 19c, and 19d are end elevational view in cross-section of four different embodiments of a guide wire positioned within the internal passage of a cannula.

FIG. 20 is a cross-sectional view, partially in phantom, of a portion of an insertion device illustrated with the deployed guide wire deployed within a curved bone tunnel.

FIGS. 21a, 21b, 21c, 21d, 21e, 21f, and 21g are plan views of seven different embodiments of a deployed guide wire disposed within a nucleus pulposus of a disc.

FIG. 22a is a cross-sectional perspective view of a portion of a first embodiment of a RF probe assembly illustrating an inflation device in a deflated condition.

FIG. 22b is a cross-sectional view, taken along the line 22b-22b of FIG. 22a.

FIG. 23a is a cross-sectional perspective view of the portion of the RF probe assembly of FIG. 22a illustrated with the inflatable device in an inflated condition.

FIG. 23b is a cross-sectional view, taken along the line 23b-23b of FIG. 23a.

FIG. 24a is a cross-sectional perspective view of a portion of a cannulated fixed RF probe assembly.

FIG. 24b is a cross-sectional perspective view of a portion of another embodiment of a cannulated fixed RF probe assembly.

FIG. 25 is a cross-sectional perspective view of a portion of another embodiment of a RF probe assembly illustrated with an external cannulated guide support.

FIG. 26 is a cross-sectional perspective view of a portion of another embodiment of a RF probe assembly illustrated with a segmented external cannulated guide support.

FIG. 27a is a cross-sectional perspective view of a portion of another embodiment of a RF probe assembly illustrated with an RF probe element having randomly-shaped cutouts.

FIG. 27b is a cross-sectional perspective view of a portion of another embodiment of a RF probe assembly illustrated with an RF probe element having triangularly-shaped cutouts.

FIG. 28a is a cross-sectional perspective view of a portion of another embodiment of a RF probe assembly illustrated with an RF probe element having randomly-shaped cutouts and a guide support having randomly-shaped cutouts.

FIG. 28b is a cross-sectional perspective view of a portion of another embodiment of a RF probe assembly illustrated with an RF probe element having triangularly-shaped cutouts and a guide support having triangularly-shaped cutouts.

FIG. 29 is a cross-sectional view of a portion of a first embodiment of a mechanical abrading assembly.

FIG. 30 is a cross-sectional view of a portion of a second embodiment of a mechanical abrading assembly having a chamfered cross-sectional shape.

FIG. 31 is a cross-sectional view of a portion of a third embodiment of a mechanical abrading assembly having a cannulated guide support.

FIG. 32 is a cross-sectional view of a portion of a fourth embodiment of a mechanical abrading assembly having a cannulated guide support and a hood.

FIG. 33 is a cross-sectional view of a portion of a fifth embodiment of a mechanical abrading assembly having a cannulated guide support and an angled portion.

FIG. 34 is a cross-sectional view of a portion of a sixth embodiment of a mechanical abrading assembly having a reamer with expandable reamer blades.

FIG. 35 is a cross-sectional perspective view of a seventh embodiment of a mechanical abrading assembly having an expandable reamer with an inflatable device positioned within the reamer.

FIG. 36 is a cross-sectional perspective view of a portion of an ablating assembly having a radiofrequency-based ablating surface.

FIG. 37 is a cross-sectional view of a first embodiment of a portal cannula.

FIG. 38 is a cross-sectional view of a second embodiment of a portal cannula having inflatable devices.

FIG. 39 is a cross-sectional view of a third embodiment of a portal cannula having an inflatable collar.

FIG. 40 is an end elevational view of the portal cannula of FIG. 39.

FIG. 41 is a side view, partially in cross section, of a first embodiment of a pedicle anchor.

FIG. 42 is a side elevational view, partially in phantom, of a disc and vertebrae illustrating a curved bone tunnel within one of the vertebra.

FIG. 43 is a side elevational view, partially in phantom, of the disc and vertebrae of FIG. 42 illustrating a first embodiment of an ultrasonic probe positioned in the nucleus pulposus of the disc.

FIG. 44 is a side elevational view, partially in phantom, of the disc and vertebrae of FIG. 42 illustrating a second embodiment of an ultrasonic probe positioned in the nucleus pulposus of the disc.

### DETAILED DESCRIPTION OF THE PREFERRED EMBODIMENTS

Referring now to the drawings, there are illustrated a variety of apparatuses and methods of accessing an intervertebral disc space for surgical procedures including, but not limited to, discectomy, end plate preparation, and interbody fusion. Generally, the apparatuses and methods provide access to a nucleus pulposus of an intervertebral disc through a vertebral end plate without disturbing the annulus fibrosus of the intervertebral disc.

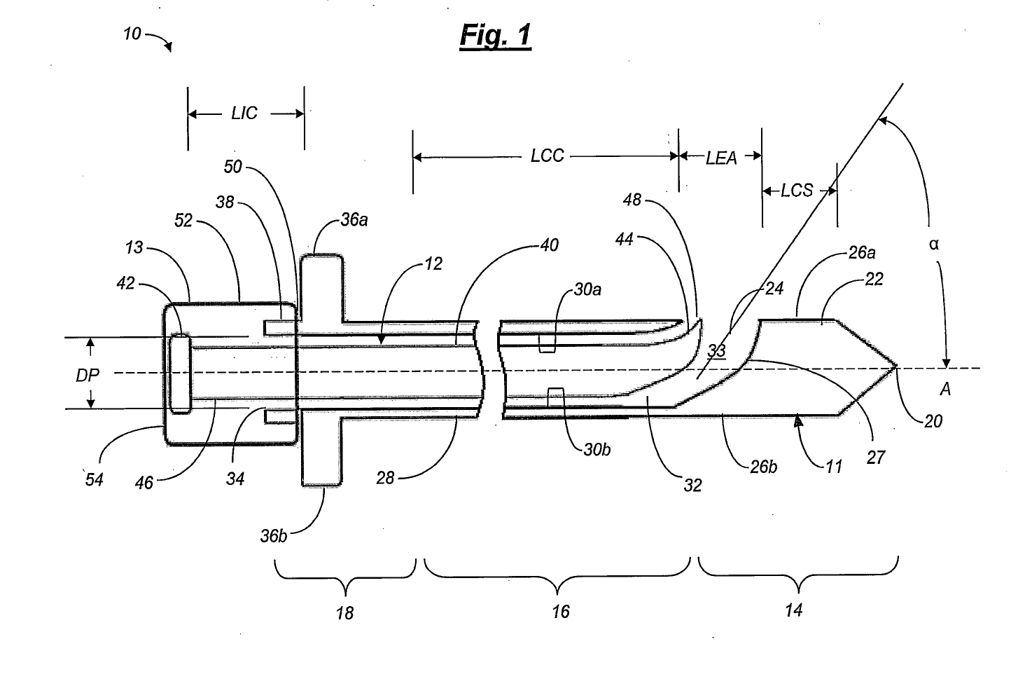

Referring now to FIGS. 1-3, there is illustrated an insertion device, indicated generally at 10, that is adapted to penetrate a vertebra through a vertebral pedicle, form a curved bone tunnel in the vertebra, and access a nucleus pulposus of an intervertebral disc located adjacent to the vertebra. In FIG. 1, the insertion device 10 is shown prior to insertion into the vertebra. The insertion device 10 includes a cannula indicated generally at 11, an obturator indicated generally at 12, and an impaction cap 13. The cannula 11 includes a distal end 14, a center portion 16 and a proximal end 18. The cannula distal end 14 includes a tip 20, a shank portion 22, and an exit aperture 24. The tip 20 is configured to penetrate a vertebral pedicle and the vertebra. In the illustrated embodiment, the tip 20 has a diamond shape. However, the tip 20 can have any other desired configuration that is suitable to penetrate the vertebral pedicle and the vertebra, such as for example a conical shape, a spear shape, a beveled shape, or a drill bit shape. In the illustrated embodiment, the tip 20 is a closed tip. Alternatively, the cannula tip 20 can have an open configuration (similar to that side elevational shown in FIG. 12).

Referring again to FIG. 1, the cannula shank portion 22 extends from the cannula tip 20 to the exit aperture 24. The cannula shank portion 22 includes opposed shank sides 26a and 26b and a deflector 27. In the illustrated embodiment, the opposed shank sides 26a and 26b are substantially parallel, although such is not required. In the illustrated embodiment, the cannula shank portion 22 has a circular cross-sectional shape. Alternatively, the cannula shank portion 22 can have any other desired cross-sectional shape. The cannula shank portion 22 has a length LCS. In the illustrated embodiment, the length LCS of the cannula shank portion 22 is approximately five mm. In other embodiments, the length LCS of the cannula shank portion 22 can be more or less than approximately five mm. The deflector 27 will be discussed in more detail below.

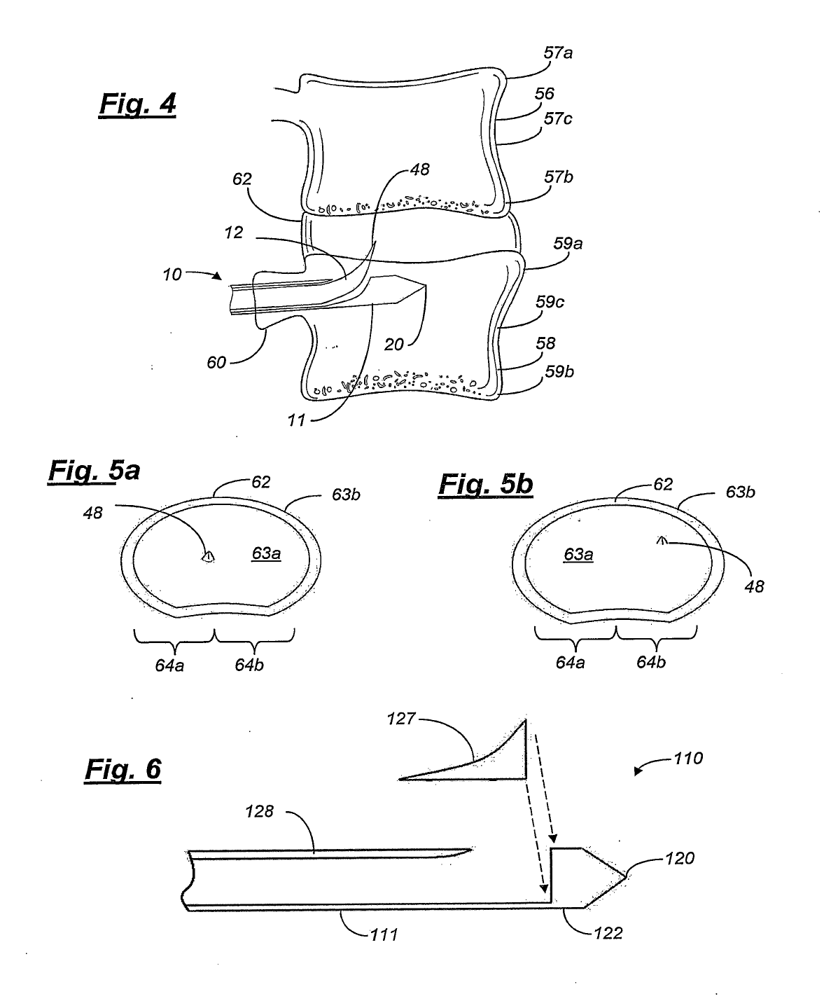

The exit aperture 24 is positioned between the cannula center portion 16 and the cannula shank portion 22. The exit aperture 24 is configured to allow one end of the obturator 12 to exit the cannula 11 and engage a vertebral end plate 59a (as seen in FIG. 4). Referring again to FIG. 1, the exit aperture 24 has a length LEA. In the illustrated embodiment, the length LEA of the exit cannula aperture 24 is approximately three mm. In other embodiments, the length LEA of the exit aperture 24 can be more or less than approximately three mm. The exit aperture 24 will be discussed in more detail below.

Referring again to FIG. 1, the cannula center portion 16 extends from the cannula distal end 14 to the cannula proximal end 18. The cannula center portion 16 has a length LCC. In the illustrated embodiment, the length LCC of the cannula center portion is in a range of from about fifty mm to about one hundred fifty mm. In other embodiments, the length LCC of the cannula center portion 16 can be more than about one hundred fifty mm or less than about fifty mm. In the illustrated embodiment, the cannula center portion 16 has a circular cross-sectional shape. Alternatively, the cannula center portion 16 can have other desired cross-sectional shapes.

The cannula center portion 16 includes an elongated tube 28. The elongated tube 28 includes opposing inner tube walls 30a and 30b. In the illustrated embodiment, the opposing inner tube walls 30a and 30b, are substantially parallel. In other embodiments, the opposing inner tube walls 30a and 30b, can be non-parallel. The opposing inner tube walls 30a and 30b define a passage 32. The passage 32 is configured to house a substantial portion of the obturator 12 and guide the obturator 12 as one end of the obturator 12 exits the insertion device 10 through the exit aperture 24. The passage 32 has a diameter DP. In the illustrated embodiment, the diameter DP of the passage 32 is approximately four mm. In other embodiments, the diameter DP of the passage 32 can be more or less than approximately four mm. The elongated tube 28 has a longitudinal axis A.

Referring again to FIG. 1, the passage 32 has a distal end 33 and a proximal end 34. The distal end 33 of the passage 32 terminates at the deflector 27. The deflector 27 is configured to guide one end of the obturator 12 as that end exits the insertion device 10 through the exit aperture 24. In the illustrated embodiment, the deflector 27 has an arcuate shape. However, the deflector 27 can have any other desired shape, including but not limited to an angled shape, that is adapted to guide one end of the obturator 12 as that end exits the insertion device 10 through the exit aperture 24. The deflector 27 forms an exit angle α with the longitudinal axis A of the elongated tube 28. In the illustrated embodiment, the exit angle α is approximately 45°. In other embodiments, the exit angle α can be more or less than approximately 45°.

Optionally, the deflector 27 can be embodied as any desired structure, mechanism, or device that is configured to facilitate advancement of the obturator 12 toward the vertebral end plate as the obturator 12 exits the insertion device 10 through the exit aperture 24. One non-limiting example of such a structure is a longitudinal groove (not shown). However other structures, mechanisms, and devices can be used.

Referring again to FIG. 1, the cannula proximal portion 18 extends from the cannula center portion 16. The cannula proximal portion 18 includes opposing cannula shoulders 36a and 36b and a cannula guide 38. The cannula shoulders 36a and 36b are configured for allowing a user to grip the insertion device 10 and to function as a contact surface for the impaction cap 13. The cannula guide 38 extends from the cannula shoulders 36a and 36b and is configured to support the impaction cap 13 as the insertion device 10 penetrates vertebra. The cannula guide 38 will be discussed in more detail below.

In the illustrated embodiment, the distal end 14, the center portion 16, and the proximal end 18 of the cannula 11 are formed from a single piece of material. However, the distal end 14, the center portion 16, and the proximal end 18 of the cannula 11 can be formed of separate components joined together in any desired manner.

Referring again to FIG. 1, the obturator 12 includes an elongated portion 40 connected to an obturator head 42. The elongated portion 40 of the obturator 12 includes a distal end 44 and a proximal end 46. The distal end 44 of the elongated portion 40 includes a probe 48. The probe 48 is configured to form a bone tunnel within the vertebra as the probe 48 exits the insertion device 10 through the exit aperture 24. In one embodiment, the probe 48 is a cutter having a cone shape. However, the probe 48 can have any other desired configuration that is adapted to form a bone tunnel within the vertebra as the probe 48 exits the insertion device 10 through the exit aperture 24.

The obturator head 42 is positioned at the proximal end 46 of the obturator 12 and is configured to urge the probe 48 through the exit aperture 24 when a force F is applied (see FIG. 3) to the obturator head 42 in a direction substantially parallel to the elongated portion 40 of the obturator 12. In the illustrated embodiment, the obturator head 42 has a substantially cylindrical shape. Alternatively, the obturator head 42 can have other desired shapes sufficient to urge the probe 48 through the exit aperture 24 as a force is applied to the head in a direction substantially parallel to the elongated portion 40 of the obturator 12.

Referring now to FIG. 3, the obturator 12 is shown in an extended position, wherein the probe 48 has been moved through the exit aperture 24. Accordingly, it can be seen that the illustrated obturator 12 is somewhat flexible. The obturator 12 may, for example, be formed from a shape memory metallic alloy having the ability to flex, such as nickel titanium or nitinol. However, the obturator 12 can be formed from any other desired material or combination of materials including, but not limited to, polyetheretherketone (“PEEK”). In still other embodiments, the obturator 12 can be made from combinations of materials such as metals, shape memory metals, and biomaterials sufficient to improve the flexibility of the obturator 12 and allow improved clearance as the obturator 12 is slidably moved along the axis A in the cannula 11 and through the formed bone tunnel. In still other embodiments, the obturator 12 can be made of other materials or structures such as, for example, an articulating structure sufficient to flex or reshape portions, as the obturator 12 exits the exit aperture 24. The obturator 12 can have any desired surface, finish or coating.

Referring again to FIG. 1, the impaction cap 13 is initially positioned over the obturator head 42 and against the cannula shoulders 36a and 36b. In this position, the impaction cap 13 is configured to urge the insertion device 10 through the pedicle and into the vertebral body while the probe 48 of the obturator 12 is housed substantially within the passage 32 of the cannula 11. Generally, after the insertion device 10 is positioned within the vertebral body in a desired location, the impaction cap 13 is removed so as to allow the probe 48 of the obturator 12 to be advanced through the exit aperture 24 of the cannula 11.

The impaction cap 13 includes a contact edge 50, a side wall 52, and an impaction end wall 54. The contact edge 50 is configured to seat against the cannula shoulders 36a and 36b as the insertion device 10 is urged through the pedicle and into the vertebral body. In the illustrated embodiment, the contact edge 50 has a substantially flat surface that corresponds to the substantially flat surface of the cannula shoulders 36a and 36b. However, the contact edge 50 can have any other desired configuration. For example, the contact edge 50 and the cannula shoulders, 36a and 36b, can have interlocking structures that lock the impaction cap 13 in position until the insertion procedure is completed.

Referring again to FIG. 1, the impaction cap 13 is positioned over the obturator head 42. The proximal end 46 of the obturator 12 extends a length LIC from the cannula shoulders 36a and 36b. In the illustrated embodiment, the length LIC is approximately twenty five mm. However, the length LIC can be more or less than approximately twenty five mm.

The side wall 52 of the impaction cap 13 extends between the contact edge 50 to the impaction end wall 54. The side wall 52 is configured to transfer force applied to the impaction end wall 54 to the contact edge 50. In the illustrated embodiment, the side wall 52 has a circular cross-sectional shape. In other embodiments, the side wall 52 can have other desired cross-sectional shapes sufficient to transfer force applied to the impaction end wall 54 to the contact edge 50.

The impaction end wall 54 is configured to receive force intended to urge the insertion device 10 through a pedicle and into the vertebral body. The force can be applied to the impaction end wall 54 in any desired manner. In the illustrated embodiment, the impaction end wall 54 has a substantially flat surface. In other embodiments, the impaction end wall 54 can have other desired surfaces. In still other embodiments, the impaction end wall 54 can be configured to mate with other mechanisms, structures and devices (not shown) for imparting a force intended to urge the insertion device 10 through a pedicle and into the vertebral body.

The impaction cap 13 can be made from any desired material or combination of materials that is sufficient to transfer force applied to the impaction cap 13 to the insertion device 10. The impaction cap 13 can have any desired surface, finish, or coating.

As described above, FIG. 1 illustrates the insertion device 10 in a pre-insertion configuration. The pre-insertion configuration includes the obturator 12 being housed substantially within the passage 32 of the cannula 11 such that the probe 48 does not extend through the exit aperture 24 of the cannula 11. The pre-insertion configuration also includes the obturator head 42 extending a distance LIC from the cannula 11. The pre-insertion configuration further includes the impaction cap 13 installed over the obturator head 42 and seated against the cannula shoulders 36a and 36b.

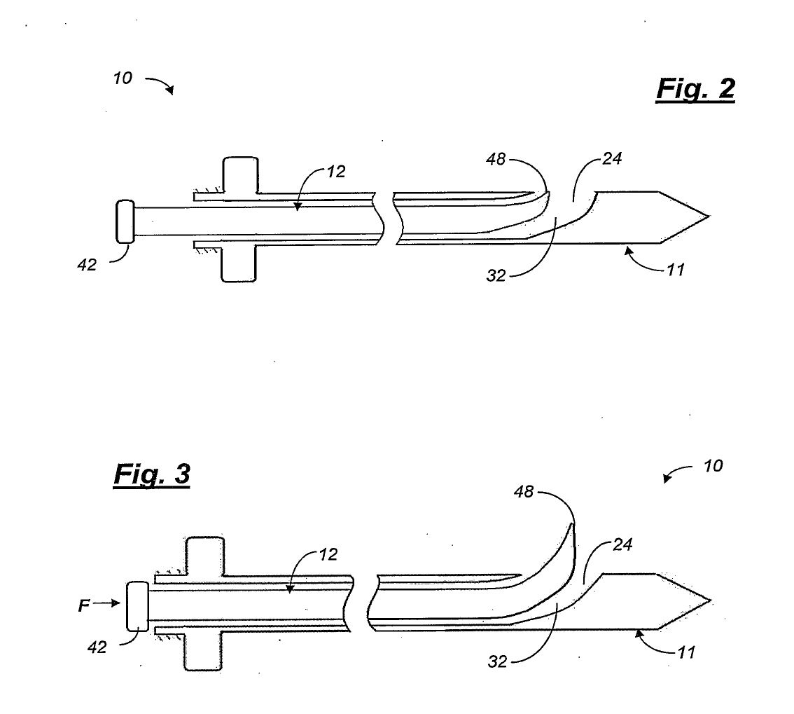

Referring now to FIG. 2, the insertion device 10 is illustrated after at least the cannula tip 20 of the insertion device 10 has been inserted through a pedicle and positioned in a corresponding vertebral body. After insertion, the impaction cap is been removed, thereby exposing the extended obturator head 42. At this time, the obturator 12 remains housed substantially within the passage 32 of the cannula 11 such that the probe 48 does not extend through the exit aperture 24 of the cannula 11. As shown in FIG. 3, force F has been applied to the obturator head 42, resulting in movement of the probe 48 through the exit aperture 24 of the cannula 11. Movement of the probe 48 beyond the exit aperture 24 results in the formation of a curved bone tunnel (not shown). It should be understood that the vertebral body is made of cancellous bone. Therefore, the amount of force that is typically necessary to penetrate the vertebral body is generally less than the amount of force that is typically necessary to penetrate the pedicle.

Referring now to FIG. 4, the use of the insertion device 10 in a surgical procedure is illustrated in connection with two spinal vertebrae 56 and 58. As shown in FIG. 4, the vertebra 56 includes an upper end plate 57a, a lower end plate 57b, and a waist portion 57c located between the upper end plate 57a and the lower end plate 57b. Similarly, the vertebra 58 includes an upper end plate 59a, a lower end plate 59b, and a waist portion 59c located between the upper end plate 59a and the lower end plate 59b. Extending from the vertebra 58 is a pedicle 60. As shown in FIG. 4, the vertebrae 56 and 58 are separated by a fibro-cartilaginous structure commonly referred to as an intervertebral disc 62.

Referring again to FIG. 4, the cannula tip 20 of the insertion device 10 is shown urged through the pedicle 60 and into the vertebra 58. Once the cannula tip 20 is forced through the pedicle 60 and in the vertebra 58, the probe 48 of the obturator 12 is deployed through the exit aperture 24 and directed toward the disc 62. The disc 62 includes a nucleus pulposus 63a surrounded by an annulus fibrosus 63b. The nucleus pulposus 63a has a left quadrant 64a and a right quadrant 64b. As shown in FIGS. 5a and 5b, the insertion device provides access to a nucleus pulposus 63a of the disc 62 without disturbing the annulus fibrosus 63b. As shown in FIG. 5a, the probe 48 of the obturator enters the nucleus pulposus 63a in the left quadrant 64a without disturbing the annulus fibrosus 63b. Similarly, FIG. 5b illustrates the probe 48 of the obturator entering the nucleus pulposus 63a in the right quadrant 64b without disturbing the annulus fibrosus 63b.

As discussed above, the cannula 11 can be formed from a single piece of material or from a plurality of separate components that are joined together. In a second embodiment shown in FIG. 6, a modified insertion device indicated generally at 110 has a cannula 111 that includes a tip 120, a shank portion 122, and an elongated tube 128. In this embodiment, a deflector 127 is provided as a separate component that is attached to the cannula 111 adjacent the cannula shank portion 122. This embodiment advantageously provides for easy replacement of deflectors 127 having different exit angles. Additionally, this embodiment provides for deflectors 127 made of different materials, such as polyetheretherketone (“PEEK”) for example, to be easily installed in the insertion device 110.

Referring now to FIGS. 7 and 8, a third embodiment of an insertion device, indicated generally at 210, is illustrated. The insertion device 210 includes a cannula 211 that includes a proximal end 246 of an obturator 212 that is positioned in a first passage 264 in the cannula 211. The first passage 264 is connected to a second passage 232 in the cannula 211. The first passage 264 defines an angle β with respect to the longitudinal axis A of an elongated tube 228 of the cannula 211. In the illustrated embodiment, the angle β is in a range of from about 15° to about 60°. However, in other embodiments the angle β can be less than about 15° or more than about 60°.

The cannula 211 includes a cannula head 267. The cannula head 267 is configured to urge the insertion device 210 through the pedicle 60 and through the vertebra 58 while a probe 248 of the obturator 212 is housed substantially within the passages 232 and 264 as described above. The cannula head 267 can have any desired shape sufficient to urge the insertion device 210 through the pedicle 60 and through the vertebra 58. In FIG. 7, the insertion device 210 is illustrated in a pre-insertion configuration, wherein the obturator 212 is disposed substantially within the passages 232 and 264 of the cannula 211 such that the probe 248 does not extend through the exit aperture 224. In this pre-insertion configuration, the obturator head 242 is spaced apart from the cannula 211. After insertion through the pedicle 60 and the vertebra 58, the obturator 212 initially remains housed substantially within the passages 232 and 264 such that the probe 248 does not extend through the exit aperture 224. Thereafter, as shown in FIG. 8, a force F is applied to the obturator head 242, resulting in movement of the probe 248 through the exit aperture 224 of the cannula 211. Movement of the probe 248 beyond the exit aperture 224 results in the formation of a curved bone tunnel as described above.

The obturator 212 can be formed from any desired material, such as shape memory metallic alloys having the ability to flex (such as, for example, nickel titanium or nitinol) while providing the probe 248 with the ability to perforate the vertebral end plate and form the curved bone tunnel within the vertebra. Alternatively, the obturator 212 can be formed from polyetheretherketone (“PEEK”), combinations of materials such as metal, shape memory metal, and biomaterials and articulating structures. The obturator 212 can have any desired surface, finish or coating.

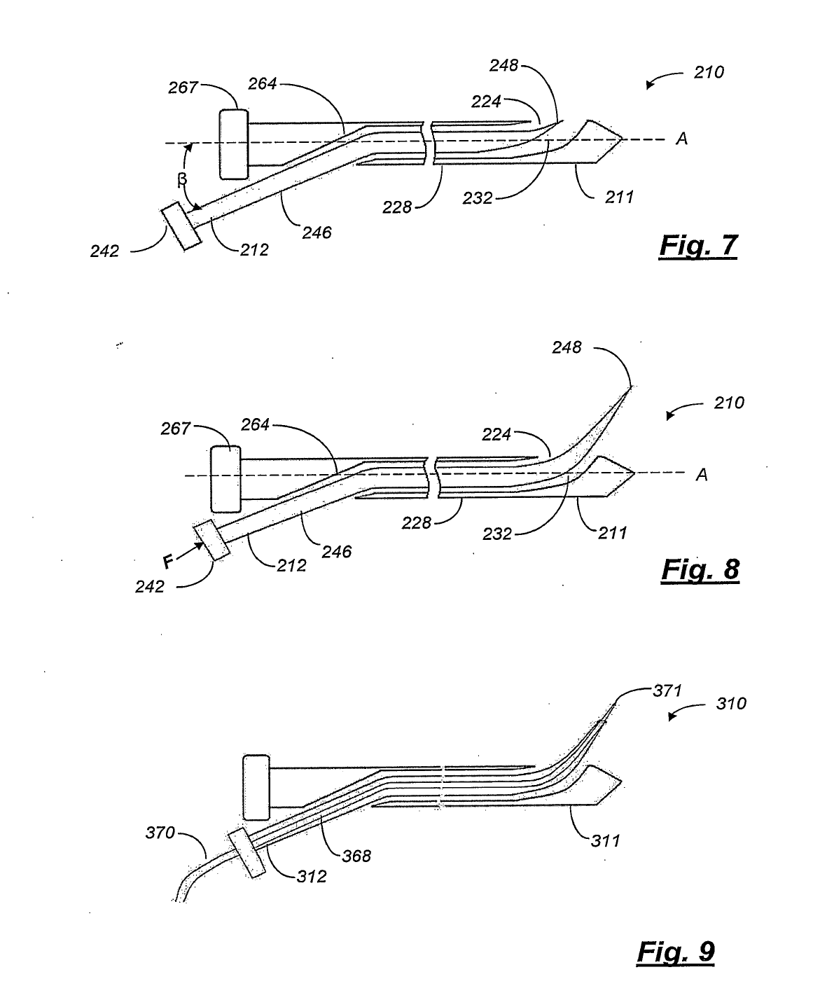

Referring now to FIG. 9, a fourth embodiment of an insertion device, indicated generally at 310, having a cannula 311 is illustrated. In this embodiment, the insertion device 310 is substantially the same as the insertion device 210 illustrated in FIGS. 7 and 8, except that it includes a modified obturator 312 that is itself cannulated, thereby defining a channel 368 within the obturator 312. A guide wire 370 can be positioned within the channel 368. In one embodiment, the guide wire 370 can have a tip 371 having a conical cross-sectional shape forming a sharp point. In other embodiments, the guide wire 370 can have other cross-sectional shapes forming rounded points. In still other embodiments, the guide wire 370 can have a threaded or unthreaded tip 371.

In one embodiment of a surgical procedure utilizing the insertion device 310, the guide wire 370 is pre-positioned within the channel 368 of the obturator 312 prior to the insertion of the insertion device 310 into the vertebra 58. In other embodiments, the guide wire 370 can be inserted into the channel 368 of the obturator 312 following insertion of the insertion device 312 into the vertebra 58.

In the illustrated embodiment, the guide wire 370 is advanced through the vertebra 58 and remains in place following the subsequent removal of the cannula 311 and the obturator 312 of the insertion device 310. In this embodiment, the guide wire 370 can be subsequently used as a guide for widening of the curved bone tunnel by any desired manner such as, for example, by tapping or drilling. Once the curved bone tunnel is widened to a desired diameter, the guide wire 370 can be removed and replaced with other guide wires, as discussed below in more detail.

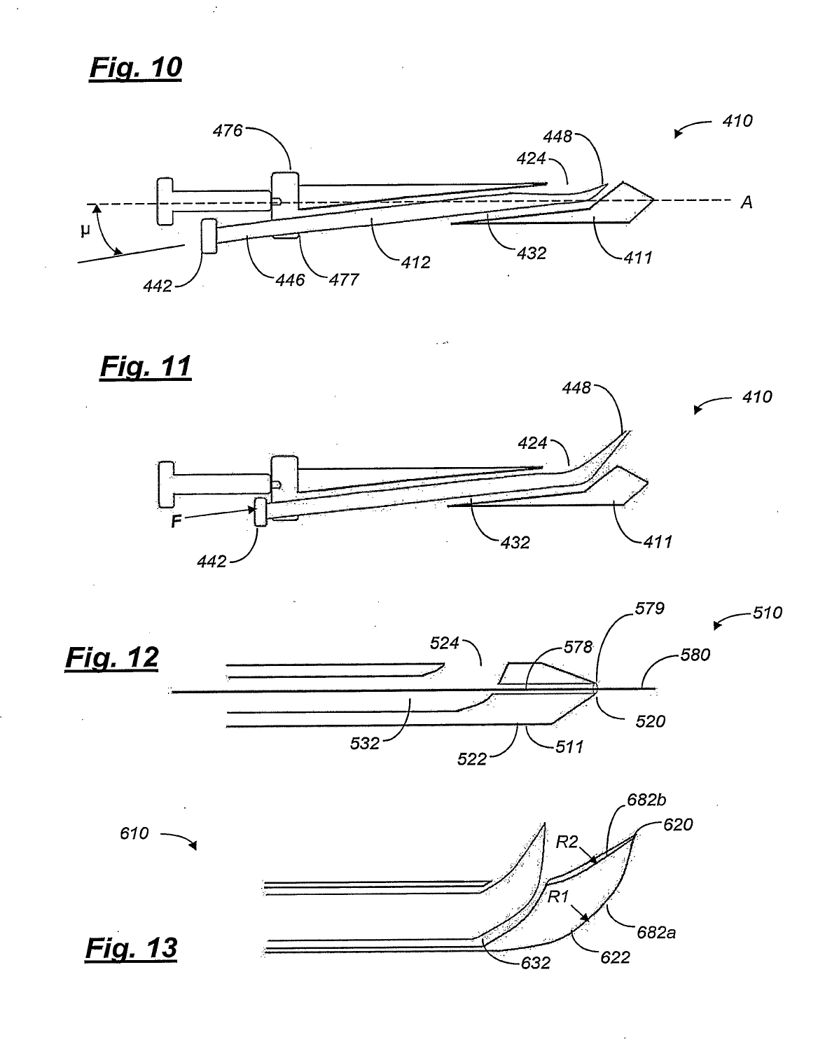

Referring now to FIGS. 10 and 11, a fifth embodiment of an insertion device, indicated generally at 410, having a cannula 411 is illustrated. In this embodiment, a passage 432 extending through the cannula 411 extends at a non-zero angle μ with respect to the axis A. An obturator 412 is substantially positioned in the angled passage 432. A proximal end 446 of the obturator 412 is supported by a flange 476. The flange 476 has a flange aperture 477 through which the obturator 412 extends. In the illustrated embodiment, the angle μ is in a range of from about 5° to about 45°. However, in other embodiments, the angle μ can be less than about 5° or more than about 45°. In the illustrated embodiment, the flange 476 has a circular cross-sectional shape. In other embodiments, the flange 476 can have other cross-sectional shapes sufficient to support the proximal end 446 of the obturator 412.

In FIG. 10, the insertion device 410 is illustrated in a pre-insertion configuration. In this pre-insertion configuration, the obturator 412 is disposed substantially within the angled passage 432 such that a probe 448 of the obturator 412 does not extend through an exit aperture 424 of the cannula 411. The pre-insertion configuration also includes the obturator head 442 extending a distance from the support 476. After insertion through the pedicle into the vertebra 58, the obturator 412 remains housed substantially within the passage 432 such that the probe 448 does not extend through the exit aperture 424. Thereafter, as shown in FIG. 11, a force F is applied to the obturator head 442, resulting in movement of the probe 448 through the exit aperture 424. Movement of the probe 448 beyond the exit aperture 424 results in the formation of a curved bone tunnel in the vertebra 58, as described above.

Referring now to FIG. 12, a sixth embodiment of an insertion device, indicated generally at 510, is illustrated. The insertion device 510 includes a guide aperture 578 extending from a passage 532 to an opening 579 of a tip 520 of the cannula 511. In one embodiment of a surgical procedure, a conventional insertion device, such as a conventional Jamshidi needle (not shown) for example, is initially inserted through the pedicle into the vertebra 58. The conventional insertion device forms a substantially straight bone tunnel. A guide wire 580 is then positioned in the formed bone tunnel, and the conventional insertion device is removed, leaving only the guide wire 580. The insertion device 510 of this invention is then inserted over the guide wire 580 until an exit aperture 524 of the insertion device 510 is positioned at a desired location within the bone tunnel. Thus, the insertion device 510 can be used to enlarge the straight portion of the bone tunnel within the pedicle 62. If desired, the insertion device 510 can include external threads (not shown) provided on a cannula shank portion 522 and configured to advance the cannula 511 within the vertebra 58 as the cannula 511 is axially rotated. Once the cannula 511 is positioned at a desired location within the vertebra 58, the cannula 511 is axially rotated such that the exit aperture 524 is facing the disc 62. Once the exit aperture 524 is positioned at the desired location, the guide wire 580 is removed and an obturator (such as described above) is urged through the exit aperture 524 and toward the disc 62, as described above.

A seventh embodiment of an insertion device, indicated generally at 610, is illustrated in FIG. 13. The insertion device 610 includes a cannula shank portion 622 extending from a tip 620 to a passage 632. In the embodiment, the cannula shank portion 622 includes an arcuate lower shank surface 682a and an arcuate upper shank surface 682b. The arcuate lower shank surface 682a defines a first radius R1 and the arcuate upper shank surface 682b defines a second radius R2. The arcuate lower shank surface 682a and the arcuate upper surface 682b allow the insertion device 610 to be inserted into the vertebra 58 at a variety of angles, thus facilitating improved entry into the disc 62. In the illustrated embodiment, the first radius R1 is approximately ten mm and the second radius R2 is approximately thirty mm. In other embodiments, the first radius R1 can be more or less than approximately ten mm and the second radius R2 can be more or less than approximately thirty mm.

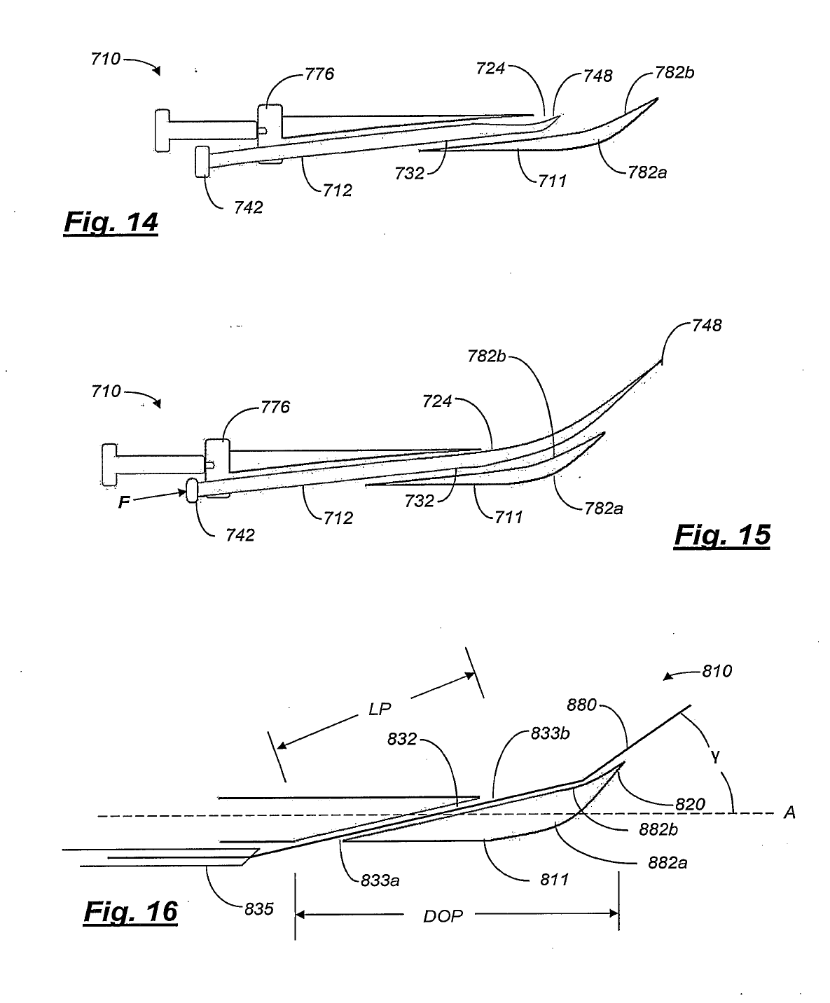

Referring now to FIGS. 14 and 15, an eighth embodiment of an insertion device 710 is illustrated. This embodiment of the insertion device 710 includes an angled passage 732 (similar to that shown in FIGS. 10 and 11) and an arcuate lower shank surface 782a and arcuate upper shank surface 782b (similar to that shown in FIG. 13). The insertion device 710 includes a cannula 711. In a pre-insertion configuration illustrated in FIG. 14, an obturator 712 is housed substantially within the angled passage 732 such that a probe 748 does not extend through an exit aperture 724. The pre-insertion configuration also includes an obturator head 742 that is spaced apart by a distance from a support 776. After insertion through the pedicle 60 and the vertebra 58, the obturator 712 remains housed substantially within the passage 732 such that the probe 748 does not extend through the exit aperture 724 of the cannula 711. As shown in FIG. 15, once the cannula 711 is positioned within the vertebra 58, a force F is applied to the obturator head 742 resulting in movement of the probe 748 through the exit aperture 724. Movement of the probe 748 beyond the exit aperture 724 results in the formation of a curved bone tunnel as described above.

A ninth embodiment of an insertion device 810 having a cannula 811 is illustrated in FIG. 16. As shown in FIG. 16, the insertion device 810 includes a passage 832 in the cannula 811, an arcuate lower shank surface 882a, and an arcuate upper shank surface 882b similar to that shown in FIGS. 14 and 15. The passage 832 is straight and oriented obliquely across the cannula 811. A first end 833a of the passage 832 is located a distance DOP from a tip 820. In the illustrated embodiment, the distance DOP is in a range of from about thirty mm to about sixty mm. In other embodiments, the distance DOP can be less than about thirty mm or more than about sixty mm. A second end 833b of the passage 832 is aligned with the arcuate upper shank surface 882b, which acts as a deflecting surface as further explained below. The passage 832 has a length LP. In the illustrated embodiment, the length LP of the passage 832 is in a range of from about twenty five mm to about forty mm. In other embodiments, the length LP of the passage 832 can be less than about twenty five mm or more than about forty mm.

After the insertion device 810 is positioned within the vertebra 58, the first end 833a of the passage 832 remains external to the vertebra 58 and the second end 833b of the passage 832 is positioned past the pedicle 60 and within the vertebra 58. Once the insertion device 810 is positioned, an external alignment guide sheath 835 is positioned adjacent the insertion device 810. In one embodiment, the external alignment guide sheath 835 has a longitudinal axis that is substantially parallel to the longitudinal axis A of the insertion device 810. The guide sheath 835 can contain an opening that is substantially aligned with the first end 833a of the passage 832. In this position, the guide sheath 835 has one end that is positioned external to the skin of the patient and is configured to provide access to the passage 832.

To maintain desired rotational alignment and orientation positions, a portion of the insertion device 810 external to the skin may have varying geometrical cross-sectional shapes, such as for example rectangular, polygonal, or triangular. A corresponding internal portion of the external alignment guide sheath 835 is slid over the insertion device 832. In one embodiment, the internal portion of the external alignment guide sheath 835 has a shape that corresponds to the shape of the insertion device 810. The internal portion of the external alignment device 832 is configured to maintain a desired spatial relationship with the passage 832 during the surgical procedure. In other embodiments, the alignment of the external alignment guide sheath 835 and the insertion device 810 can be maintained by other desired devices or structures, such as for example, pins, or dovetails. In still other embodiments, the insertion device 810 can include other devices, structures, or mechanisms (not shown) configured to provide that the external alignment guide sheath advances to the desired depth and aligns with the passage 832. Non-limiting examples of other devices, structures, and mechanisms can include positive stops or locks. Optionally, a guide wire 880 can be pre-positioned substantially within the passage 832 and maintained within the insertion device 810 until advancement of the guide wire 880 is desired.

As shown in FIG. 16, as the guide wire 880 is advanced, the guide wire 880 deflects off the surface 882b and forms an angle γ with the axis A. In the illustrated embodiment, the angle γ is in a range of from about 10° to about 70°. Alternatively, the angle γ can be less than about 10° or more than about 70°. Optionally, the deflecting surface 882b can have a longitudinal groove (not shown) to facilitate advancement of the guide wire 880 in the desired trajectory.

Referring now to FIG. 17, a tenth embodiment of an insertion device 910 is illustrated. In this embodiment, an obturator 911 includes a guide wire passage 986. The guide wire passage 986 extends from an obturator head 942 in an upwardly angled direction toward a distal end 944 of the obturator 911. In the illustrated embodiment, the guide wire passage 986 extends in a substantially straight line. In other embodiments, the guide wire passage 986 can extend in an arcuate or in a multiple-arcuate manner. Optionally, the deflecting surface (not shown) of the cannula (not shown) can have a longitudinal groove (not shown) to facilitate advancement of the guide wire in the desired trajectory.

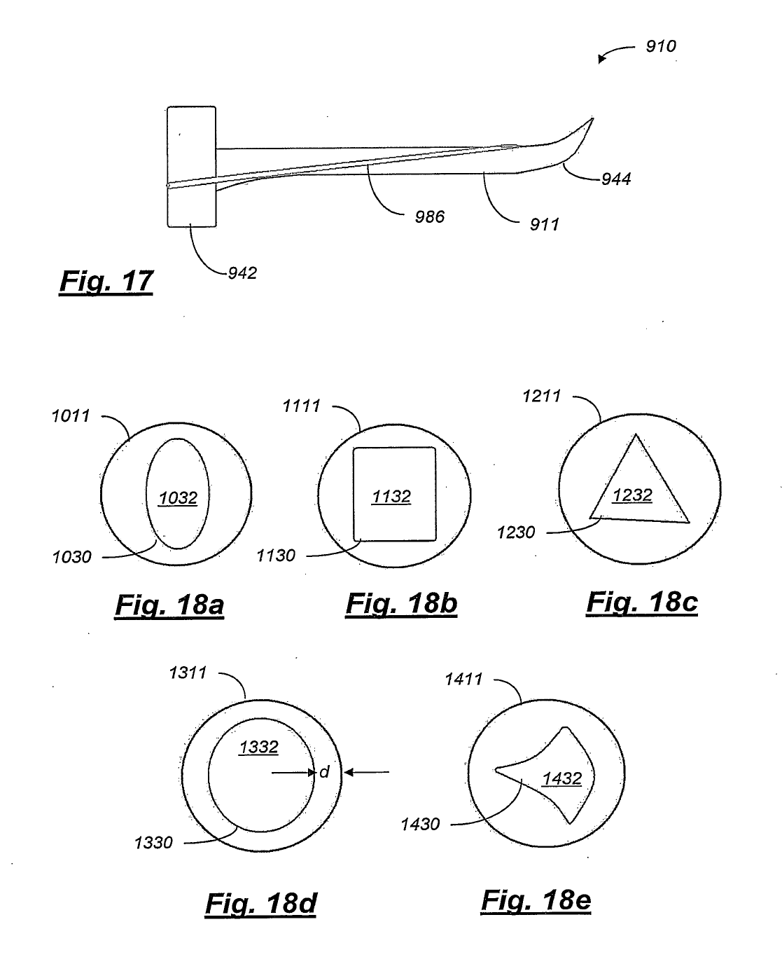

Referring now to FIGS. 18a-18e, other embodiments of various cannulae are illustrated. In the embodiments shown in FIGS. 18a-18e, an inner tube wall of the cannula forms a passage having various cross-sectional shapes. The various cross-sectional shapes of the passages are useful with obturator (not shown) having various cross-sectional shapes. In FIG. 18a, a cannula 1011 has an inner tube wall 1030 that forms a passage 1032 having a generally oval cross-sectional shape. In FIG. 18b, a cannula 1111 has an inner tube wall 1130 that forms a passage 1132 having a generally rectangular cross-sectional shape. In FIG. 18c, a cannula 1211 has an inner tube wall 1230 that forms a passage 1232 having a generally triangular cross-sectional shape. In FIG. 18d, a cannula 1311 has an inner tube wall 1330 that forms a passage 1332 having a generally circular cross-sectional shape. Finally, in FIG. 18e, a cannula 1411 has an inner tube wall 1430 that forms a passage 1432 having a generally polygonal cross-sectional shape. In each of these embodiments the passage can be oriented in any desired direction relative to the cannula.

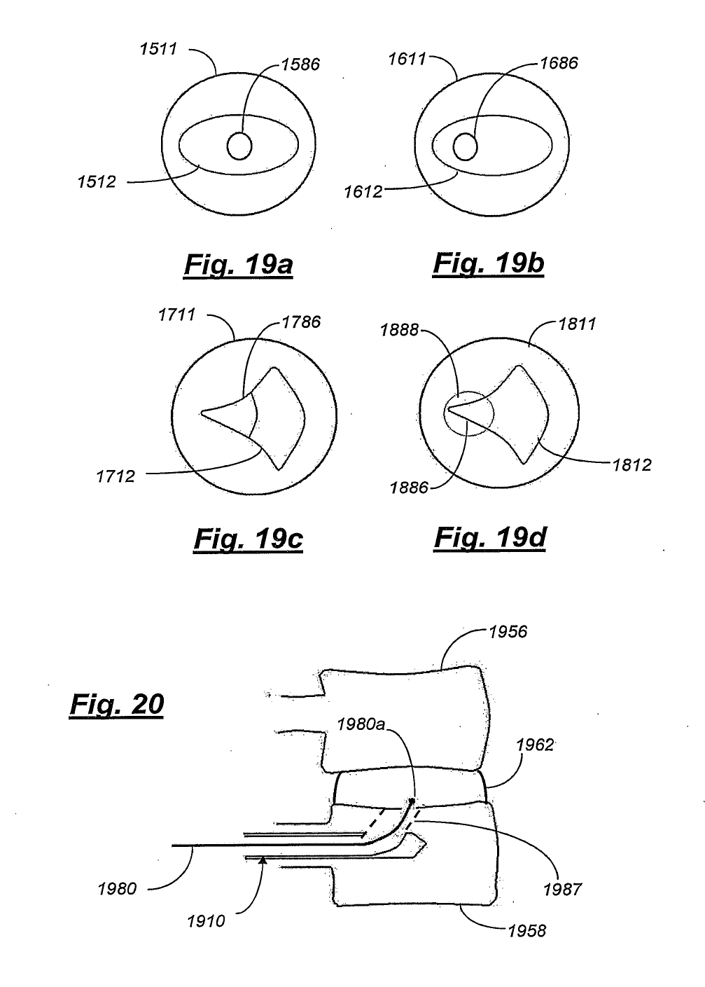

In FIGS. 19a-19d, other embodiments of various obturators are illustrated. In the embodiments shown in FIGS. 19a-19d, the obturators include a guide wire passage that can be positioned centrally or eccentrically with respect to the cross-sectional shape of the obturator. In FIG. 19a, an obturator 1512 within a cannula 1511 has a generally oval cross-sectional shape. The obturator 1512 forms a guide wire passage 1586 that is positioned centrally with respect to the cross-sectional shape of the obturator 1512. In FIG. 19b, an obturator 1612 within a cannula 1611 has a generally oval cross-sectional shape. The obturator 1612 forms a guide wire passage 1686 that is positioned in an offset or eccentric location with respect to the cross-sectional shape of the obturator 1612. In FIG. 19c, an obturator 1712 within a cannula 1711 has a generally polygonal cross-sectional shape. The obturator 1712 forms a guide wire passage 1786. As shown in FIG. 19c, the guide wire passage 1786 is positioned within a desired quadrant with respect to the polygonal cross-sectional shape of the obturator 1712. It should be understood that the guide wire passage 1786 can be positioned in other desired quadrants of the polygonal cross-sectional shape of the obturator 1712. In FIG. 19d, an obturator 1812 within a cannula 1811 also has a generally polygonal cross-sectional shape. However, in this embodiment, the obturator 1812 is removed from the cannula 1811 prior to the insertion of a guide wire (not shown). After the obturator 1812 is removed, a guide member 1888 is inserted into the cannula. The guide member 1888 includes a guide wire passage 1886 configured for guiding a guide wire (not shown).

In other embodiments, the insertion device may be inserted with the starting point slightly inferior and lateral. In these embodiments, the cannula tip can be aimed slightly cephalad. This orientation can be particularly effective in patients with large pedicles providing a greater safe zone and for cannulation of an S1 pedicle. In the event the pedicles are small, sclerotic, or fractured, it may be advantageous to utilize an extrapedicular approach to form a curved bone tunnel.

Referring now to FIG. 20, another embodiment is illustrated. In this embodiment, an insertion device 1910 has been positioned within a vertebra 1958. The obturator (not shown) has formed a curved bone tunnel 1987 in the vertebra 1958 to access a disc 1962. Optionally, the curved bone tunnel 1987 can be enlarged to provide a larger access to the nucleus pulposus. Enlarging the curved bone tunnel 1987 can be accomplished by any desired method including, but not limited to, taps or reamers. In one embodiment, the curved bone tunnel 1987 is enlarged to a diameter of about ten mm. In other embodiments, the curved bone tunnel 1987 can be enlarged to a diameter of more or less than approximately ten mm. In another optional embodiment, the curved bone tunnel 1987 can include a chamfered area (not shown) at the location where the curved bone tunnel 1987 exits the vertebra. The chamfered area is configured to prevent an acute turn of the guide wire 1980a and subsequent tools (not shown) that may be used for the discectomy and the end plate preparation as discussed in more detail below.

Referring again to FIG. 20, the guide wire 1980 has been advanced through the insertion device 1910 to the disc 1962. The guide wire 1980 can be made of polymer, metal, or shape memory material such as nitinol. The guide wire 1980 can have any desired coating or insulation. If desired, the guide wire 1980 can be a solid wire or a braided wire. In other embodiments, the guide wire 1980 can be configured for use with radio frequency disc ablators (not shown). The guide wire 1980 includes a beaded tip 1980a. The beaded tip 1980a is configured to assist the guidance the guide wire 1980 as the guide wire 1980 advances through the insertion device 1910 and into the disc 1962.

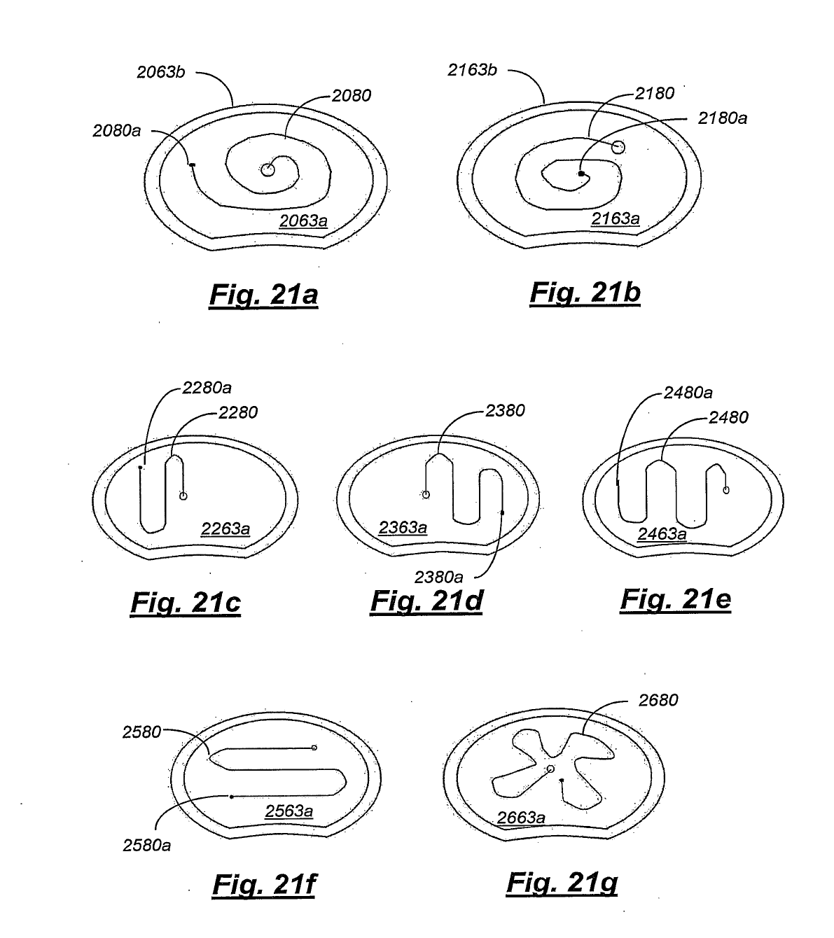

Referring now to FIGS. 21a-21g, the guide wire may assume a pre-configured shape within the nucleus pulposus. As shown in FIG. 21a, a guide wire 2080 can assume a generally spiral shape, with the spiral originating in the center of a nucleus pulposus 2063a and a beaded tip 2080a spiraling outwardly while maintaining a distance from an annulus fibrosus 2063b. As shown in FIG. 21b, a guide wire 2180 can assume a generally spiral shape, with the spiral originating in an outward region of a nucleus pulposus 2163a and a beaded tip 2180a spiraling inwardly toward the center of a nucleus pulposus 2163a while maintaining a distance from an annulus fibrosus 2163b. As shown in FIG. 21c, a guide wire 2280 can assume a grid pattern, with the grid originating near the center of a nucleus pulposus 2263a and a beaded tip 2280a extending to a side of the nucleus pulposus 2263a. Similarly as shown in FIG. 21d, a guide wire 2380 can assume a grid pattern, with the grid originating near the center of a nucleus pulposus 2363a and a beaded tip 2380a extending to a side of the nucleus pulposus 2363a. Referring now to FIG. 21e, a guide wire 2480 can assume a grid pattern, with the grid originating near one end of a nucleus pulposus 2463a and a beaded tip 2480a extending to the other end of the nucleus pulposus 2463a. As shown in FIG. 21f, a guide wire 2580 can assume a grid pattern, with the grid originating near the top of a nucleus pulposus 2563a and a beaded tip 2580a extending to the bottom of the nucleus pulposus 2563a. It should be understood that the grid pattern could originate near the bottom of the nucleus pulposus 2563a and extend to the top of the nucleus pulposus 2563a. Lastly, as shown in FIG. 21g, a guide wire 2680 can assume a generally polygonal shape, with the polygonal shape originating at any location within a nucleus pulposus 2663a.

Once a guide wire has been positioned in a desired pattern within the nucleus pulposus, a surgical procedure involving the removal of the nucleus pulposus (a “discectomy”) can be performed. The procedure can involve various medical implements, guided by the guide wire, for removal of the nucleus pulposus. As will be explained in more detail below, non-limiting examples of medical implements used for a discectomy can include mechanical implements such as disc abraders and disc shavers.

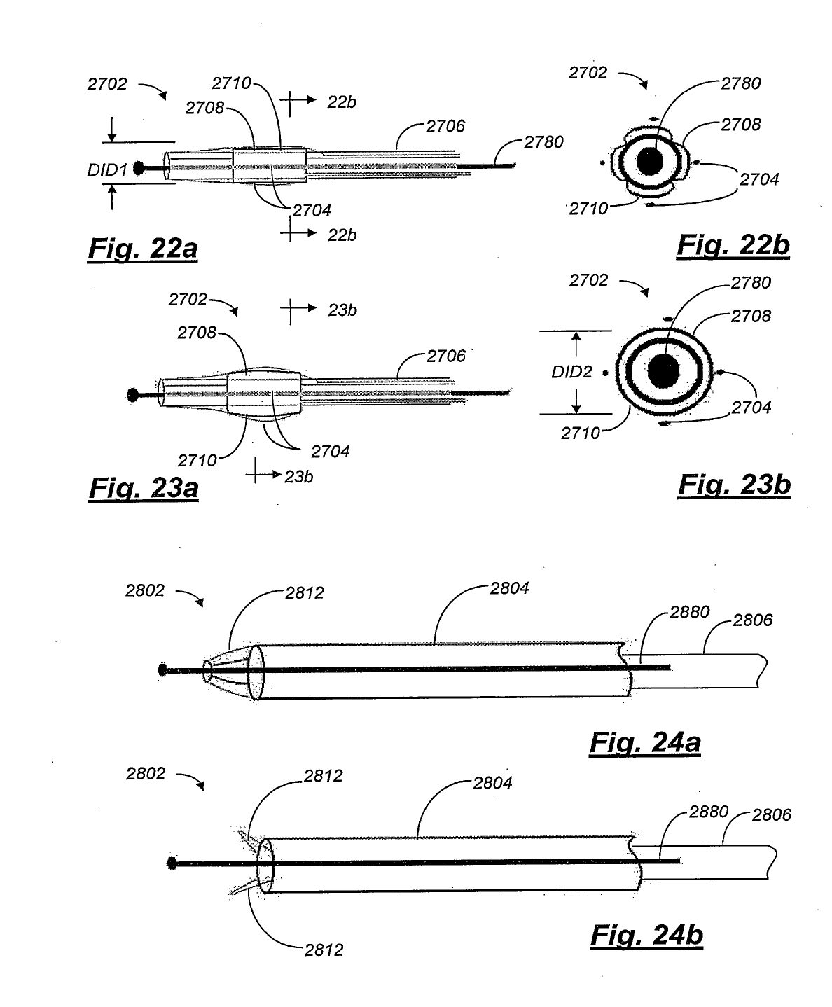

Referring now to FIGS. 22a, 22b, 23a, and 23b, an example of a medical implement that can be used to perform a discectomy is illustrated. In this embodiment, an expandable radio frequency (RF) probe assembly 2702 is used for removal of the nucleus pulposus. The RF probe assembly 2702 includes a plurality of RF filaments 2704 connected to a service cable 2706. The RF filaments 2704 are configured to deliver RF waves at a desired frequency to the nucleus pulposus (not shown). The RF waves advantageously operate to ablate the nucleus pulposus at low thermal temperatures, thereby allowing the nucleus pulposus to be removed without damage to the annulus fibrosus and surrounding tissues. In the illustrated embodiment, the RF waves can operate at a thermal temperature in a range of from about 40° F. to about 70° F. Alternatively, the RF waves can operate at other desired thermal temperatures. The RF filaments 2704 are provided RF waves by conductors (not shown) positioned within the service cable 2706. Any desired conductors sufficient to provide RF waves to the RF filaments 2704 can be used. As shown in FIG. 22a, the RF probe assembly 2702 is guided within the nucleus pulposus by a guide wire 2780. The guide wire 2780 can be the same or similar to the guide wire 580 described above and shown in FIG. 12.

An expandable inflation device 2708 is positioned adjacent a portion of the RF probe assembly 2702. In the illustrated embodiment, the expandable inflation device 2708 is positioned substantially around the RF probe assembly 2702. Alternatively, the expandable inflation device 2708 can be positioned in any desired location relative to the RF probe assembly 2702. Optionally, the inflation device 2708 can be covered with an insulating material 2710 that is sufficient to protect against thermal damage. In operation, the inflation device 2708 is configured to be deflated as the RF probe assembly 2702 is positioned within the nucleus pulposus. The inflation device 2708 has a deflated diameter DID1. In the illustrated embodiment, the deflated diameter DID1 of the deflated inflation device 2708 is approximately five mm. In other embodiments, the deflated diameter DID1 of the deflated inflation device 2708 can be more or less than approximately five mm. Once the RF probe assembly 2702 achieves the desired position within the nucleus pulposus, the expandable inflation device 2708 is inflated. The inflation device 2708 has an inflated diameter DID2. In the illustrated embodiment, the inflated diameter DID2 of the inflated inflation device 2708 is approximately fourteen mm. In other embodiments, the inflated diameter DID2 of the inflated inflation device 2708 can be more or less than approximately fourteen mm.

Inflation of the expandable inflation device 2708 is configured to maximize contact with the nucleus pulposus material to be ablated. During the discectomy, a back and forth motion of the RF probe assembly 2702 can be used to ablate the nucleus pulposus material without having to change the RF probe assembly 2702 with different probe sizes after every ablating pass. While the expandable inflation device 2708 is illustrated in FIG. 23a as expanding in a symmetric manner, it should be understood that the expandable inflation device 2708 can expand in an asymmetric manner.

As shown in FIGS. 22b and 23b, the RF filaments 2704 are positioned in a symmetric manner on the exterior of the RF probe assembly 2702. In other embodiments, the RF filaments can be positioned on the exterior of the RF probe assembly 2702 in an asymmetric manner. Configuring the RF filaments 2704 in an asymmetric manner may provide the RF probe assembly 2702 with the ability to ablate nucleus pulposus material in a direction away from the annulus fibrosus. Additionally, while the embodiments shown in FIGS. 22b and 23b have a quantity of four RF filaments 2704, it should be understood that more or less than four RF filaments 2704 can be used. While the embodiments shown in FIGS. 22a, 22b, 23a and 23b illustrate the RF filaments 2704 as having the same generally oval cross-sectional shape, it should be understood that the RF filaments 2704 can be any desired cross-sectional shape and that the cross-sectional shapes of one or more of the RF filaments 2704 can be different from the other RF filaments 2704.

Referring again to FIGS. 22a and 23a, the expandable inflation device 2708 is provided with an inflating fluid (not shown) that is adapted to inflate the inflation device 2708. In the illustrated embodiment, the inflating fluid is a saline solution. Alternatively, the inflating fluid can be other desired biocompatible fluids or solutions. In still other embodiments, the inflating fluid can contain other desired ingredients, such as for example radio-opaque dye materials. The inflating fluids can be provided to the inflation device 2708 through conductors (not shown) positioned within the service cable 2706.

While the RF probe assembly 2702 illustrated in FIGS. 22a, 22b, 23a, and 23b are shown as having an expandable diameter, in other embodiments the RF probe assemblies can have a fixed diameter and have a central lumen for passage over a guide wire. The fixed diameter RF probe assemblies can have different diameters and can be exchanged for RF probe assemblies having larger diameters after each completed ablating pass.

Referring now to FIGS. 24a and 24b, another embodiment of a RF probe assembly 2802 is illustrated. As shown in FIG. 24a, a fixed diameter RF probe assembly 2802 includes RF probe element 2804 having a plurality of RF filaments 2812 configured in a first position. While FIG. 24a illustrates a quantity of four RF filaments 2812, it should be understood that more than or less than four RF filaments 2812 can be used. As shown in FIG. 24b, the fixed diameter RF probe assembly 2802 includes RF probe elements 2804 having a plurality of RF filament loops 2812 configured in a second outward orientation. The outward orientation of the RF filament loops 2812 is configured to improve the contact of the RF filament loops 2812 with the nucleus pulposus material. While FIG. 24b illustrates a quantity of two RF filaments 2812, it should be understood that more than or less than two RF filaments 2812 can be used. While the RF filament loops 2812 are illustrated as being positioned in the front of the RF element 2804, in other embodiments the RF filament loops can be positioned at any desired location along the length of the RF element 2804 or at the rear of the RF element 2804. The RF filaments are provided power through the service cable 2806. While the embodiment shown in FIGS. 24a and 24b illustrates the fixed diameter RF probe assemblies 2802 having a central lumen configured to pass over a guide wire 2880, it should be understood that the RF probe assemblies 2802 can have any desired configuration of RF filaments 2812 and can have any desired lumen configuration.

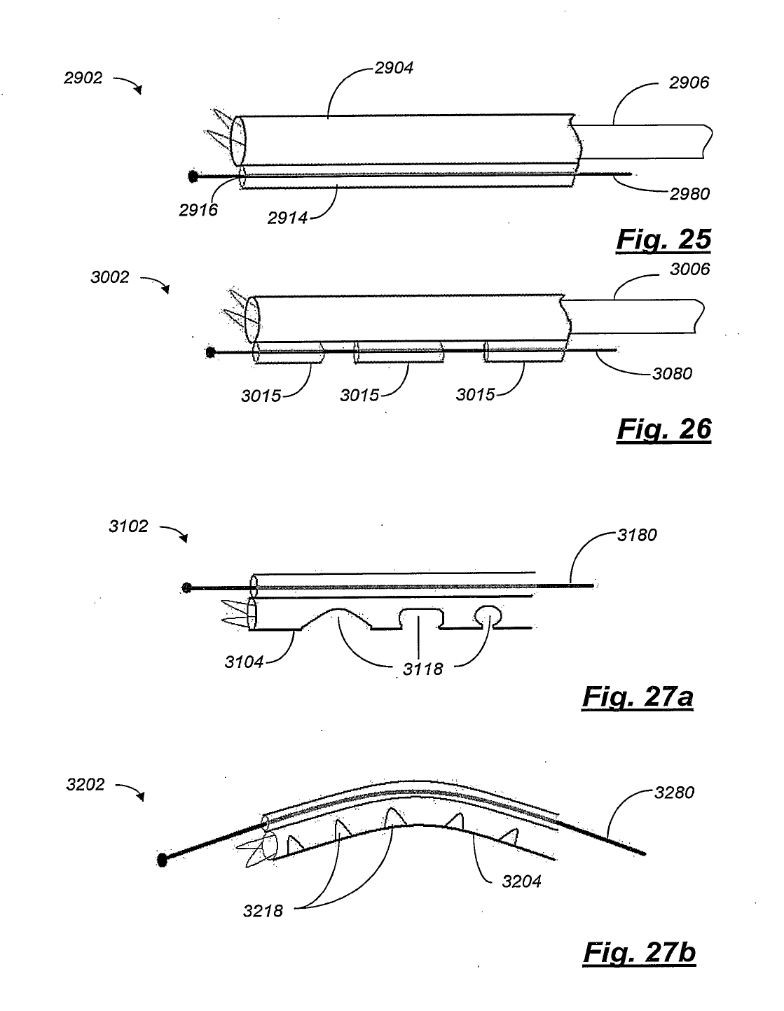

Referring now to FIG. 25, another embodiment of a RF probe assembly 2902 is illustrated. The RF probe assembly 2902 is the substantially the same as the RF probe assembly 2802 illustrated in FIG. 24a, with the exception that the RF probe assembly 2902 includes an external cannulated guide support 2914. The guide support 2914 is attached to a RF probe element 2904 and includes a support passage 2916. The guide support 2914 is configured such that a guide wire 2980 is positioned within the guide support 2914 as the RF probe assembly 2902 moves along and is guided by the guide wire 2980. In the illustrated embodiment, the guide support 2914 is made of a flexible bio-compatible material, such as a polymer. However, the guide support 2914 can be made of a bio-compatible material, an insulated bio-compatible material, or any other desired material. The guide support 2914 can have any desired length and can be attached to the RF probe assembly 2902 is any desired manner. In the embodiment illustrated in FIG. 25, the guide support 2914 is a continuous member.

Alternatively as shown in FIG. 26, a RF probe assembly 3002 can include a plurality of external cannulated guide support sections 3015. The plurality of guide support sections 3015 are configured to allow the RF probe assembly 3002 to more easily follow a bend in a guide wire 3080. As discussed above, the RF filaments are provided power through the service cable 3006.

Referring now to FIGS. 27a and 27b, other embodiments of fixed diameter RF probe assemblies are illustrated. The RF probe assemblies 3102 and 3202 are substantially the same as the RF probe assembly 2902 illustrated in FIG. 25, with the exception that RF probe assemblies 3102 and 3202 include a plurality of cutouts, 3118 and 3218 positioned in RF probe elements 3104 and 3204, respectively. The plurality of cutouts 3118 and 3218 are configured to allow the RF probe elements 3104 and 3204, respectively, to flex and more easily follow a bend in guide wires 3180 and 3280. In the embodiment shown in FIG. 27a, the cutouts 3118 have various shapes, including an arcuate shape, a rectangular shape, and a circular shape. In the embodiment shown in FIG. 27b, the cutouts 3218 have a triangular shape. In other embodiments the cutouts, 3118 and 3218, can have any desired shape sufficient to allow the RF probe elements 3104 and 3204 to flex and more easily follow a bend in the guide wires 3180 and 3280.

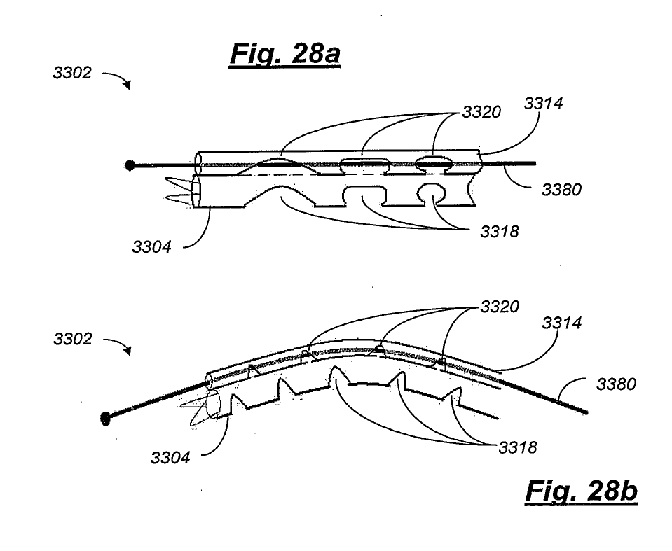

Referring now to FIGS. 28a and 28b, other embodiments of RF probe assembly 3302 are illustrated. The RF probe assembly 3302 includes a RF probe element 3304 and a guide support 3314. Generally, the RF probe assembly 3302 includes cutouts in both the RF probe element 3304 and the guide support 3314. FIG. 28a illustrates differently shaped cutouts 3318 in the RF probe assembly 3302 and the guide support 3314. FIG. 28b illustrates triangularly shaped cutouts 3318 in the RF probe assembly 3302 and the guide support 3314. The cutouts 3318 and 3320 are configured to allow the RF probe element 3304 and the guide support 3314 to flex and more easily follow a bend in a guide wire 3380. As discussed above, the cutouts 3318 and 3320 can have any desired shape sufficient to allow the RF probe element 3304 and the guide support 3314 to flex and more easily follow a bend in the guide wire 3380.

Referring again to FIGS. 27a, 27b, 28a, and 28b, the cutouts, 3118, 3218, 3318, and 3320 are configured to cause a differential stiffness in the RF probe assemblies 3102, 3202, and 3302. The differential stiffness in the RF probe assemblies, 3102, 3202, and 3302 is configured to permit preferential orientation of the RF probe assemblies 3102, 3202, and 3302, thereby allowing the RF probe assemblies 3102, 3202, and 3302 to follow a guide wire along its internal surface toward the center of the disc and away from the peripheral aspect of the guide wire and minimizing the risk of contact of the RF probe assemblies 3102, 3202, and 3302 with the annulus fibrosus. In other embodiments, the differential stiffness of the RF probe assemblies 3102, 3202, and 3302 can also be affected by other structures or devices, including the non-limiting examples of external projections, grooves, ridges, or ribs. In some embodiments, the structures or devices can be contiguous. Alternatively, the structures or devices can be noncontiguous.

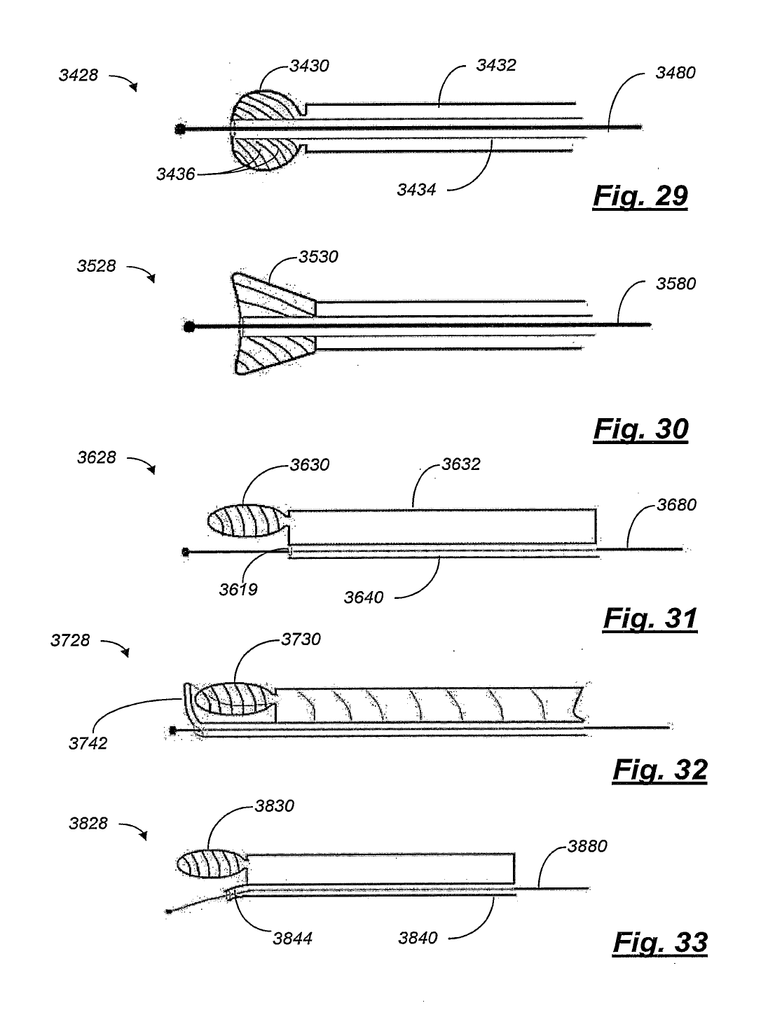

As discussed above, the discectomy (removal of the nucleus pulposus) can be performed using various medical implements guided by the guide wire. FIGS. 29 through 33 illustrate various configurations of mechanical disc abrading assemblies configured for removing the nucleus pulposus.

Referring now to FIG. 29, a first abrading assembly 3428 is illustrated. The first abrading assembly 3428 includes an abrading head 3430 attached to an actuator 3432. The abrading head 3430 and the actuator 3432 include an assembly passage 3434. The first abrading assembly 3428 is guided within the nucleus pulposus by a guide wire 3480. The guide wire 3480 can be the same as the guide wire 580 described above. The actuator 3432 is configured to rotate the abrading head 3430 at a desired rotational speed. In the illustrated embodiment, the rotational speed of the abrading head 3430 is in a range of from about 5,000 rpm to about 15,000 rpm. However, the rotational speed of the abrading head 3430 can be less than about 5,000 rpm or more than about 15,000 rpm. In the illustrated embodiment, the actuator 3432 is a rotating shaft. In other embodiments, the actuator 3432 can be other desired structures, mechanisms, and devices sufficient to rotate the abrading head 3430 at a desired rotational speed. As shown in FIG. 29, the abrading head 3430 includes abrading edges 3436 arranged in an upward orientation and having the illustrated spacing between the abrading edges 3436. It should be appreciated that the abrading edges 3436 can be arranged in any desired orientation and can have any desired spacing.

As shown in FIG. 29, the abrading head 3430 has a circular cross-sectional shape. However, the abrading head can have any other desired cross-sectional shapes. For example, as shown in FIG. 30, a second embodiment of an abrading assembly 3528 includes an abrading head 3530 having a chamfered cross-sectional shape. The chamfered cross-sectional shape of the abrading head 3530 is configured to allow the abrading head 3530 to avoid an acute bend of a guide wire 3580 or other discectomy tools (not shown) as the guide wire 3580 exits the curved bone tunnel and enters the disc space. In other embodiments, the abrading assembly 3528 can have an abrading head 3530 with a reverse chamfered cross-sectional shape. In FIG. 31, another embodiment of an abrading head assembly 3628 is illustrated. Generally, the abrading head assembly 3628 is substantially the same as the abrading head assembly 3428 illustrated in FIG. 29, with the exception that the abrading head assembly 3628 includes a cannulated guide support 3640. The guide support 3640 is attached to an actuator 3632 and includes a support passage 3619. The guide support 3640 is configured such that a guide wire 3680 is positioned within the guide support 3640 as the abrading head 3630 moves along and is guided by the guide wire 3680. In the illustrated embodiment, the guide support 3640 is made of a flexible bio-compatible material, such as a polymer. However, the guide support 3640 can be made of any other desired material. The guide support 3640 can have any desired length and can be attached to the actuator 3632 in any desired manner. In the embodiment illustrated in FIG. 31, the guide support 3640 is a continuous member. However, the guide support 3640 can be a series of discontinuous members.

Referring now to FIG. 32, another embodiment of an abrading head assembly 3728 is illustrated. In this embodiment, the abrading head assembly 3728 is substantially the same as the abrading head assembly 3628 illustrated in FIG. 31, with the exception that an abrading hood 3742 covers a portion of the abrading head 3730. The abrading hood 3742 is configured such that only a portion of the abrading head 3730 is exposed for abrading purposes. The abrading hood 3742 is configured such that unintentional damage to surrounding structures, such as the annulus fibrosus, can be substantially prevented by aligning the exposed cutting surface of the abrading head assembly 3728 away from the annulus fibrosus. The abrading hood 3742 can be made of any desired bio-compatible materials and can have any desired finish or coatings.

Referring now to FIG. 33, another embodiment of the abrading head assembly 3828 is illustrated. In this embodiment, the abrading head assembly 3828 is substantially the same as the abrading head assembly 3628 illustrated in FIG. 31, with the exception that a guide support 3840 includes an angled portion 3844 positioned beneath an abrading head 3830. The angled portion 3844 of the guide support 3840 is configured to extend the abrading head 3830 a desired distance from the guide wire 3880 as the abrading head 3830 moves along a guide wire 3880. In the illustrated embodiment, the angled portion 3844 of the guide support 3840 can be made of the same bio-compatible materials as the guide support 3840. In other embodiments, the angled portion 3844 can be made of materials different from the guide support 3840 and can have any desired finish or coatings. The angled portion 3844 can extend any desired distance and form any desired angle with the guide wire 3830.

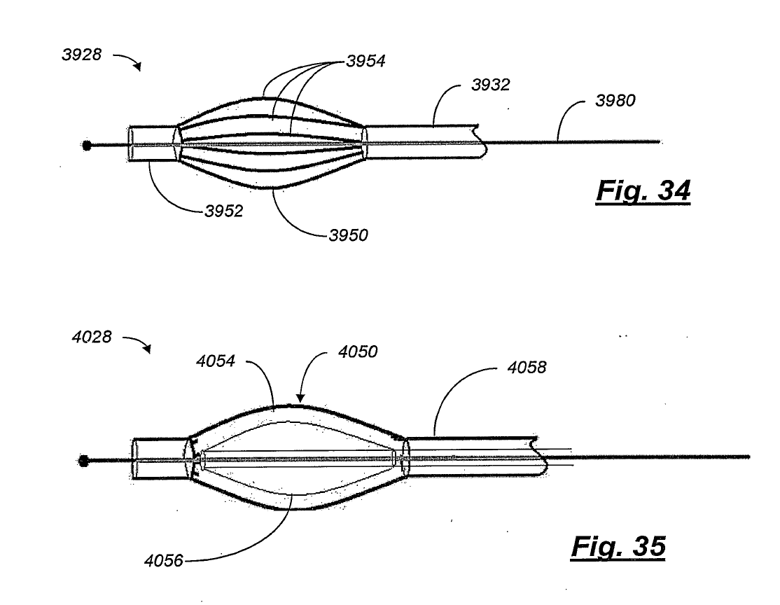

Referring now to FIG. 34, another embodiment of an expandable abrading head assembly 3928 is illustrated. The abrading head assembly 3928 includes a reamer 3950 positioned between a cannulated leading edge 3952 and an actuator 3932. The reamer 3950 includes a plurality of reamer blades 3954. In the illustrated embodiment, the reamer blades 3954 are made of shape memory metallic alloys, such as for example nitinol. In other embodiments, the reamer blades 3954 can be made of any desired bio-compatible material and have any desired finish or coating. The reamer blades 3954 are configured to expand at body temperature. The reamer blades 3954 are further configured to be rotated by the actuator 3932, thereby abrading the nucleus pulposus as the abrading head assembly 3928 moves along a guide wire 3980. The reamer blades 3954 can be flexed thereby expanding outwardly the abrading width of the abrading head assembly 3928. The reamer blades 3954 can be flexed as desired. Optionally, the reamer 3950 can be deployed with an outer cannula (not shown). The outer cannula is configured to maintain the reamer 3950 in a collapsed condition as the abrading head assembly 3928 is deployed into the nucleus pulposus. The outer cannula (not shown) can be any desired bio-compatible material sufficient to protect the reamer 3950 as the abrading head assembly 3928 is deployed into the nucleus pulposus. Once in the desired location, the optional outer cannula is retracted, thereby exposing the reamer blades 3954.

Referring now to FIG. 35, another embodiment of an abrading head assembly 4028 is illustrated. Generally, the abrading head assembly 4028 is substantially the same as the abrading head assembly 3928 illustrated in FIG. 34, with the exception that the abrading head assembly 4028 includes an inflating device 4056 positioned within reamer blades 4054 of a reamer 4050. For purposes of clarity, only a few of the reamer blades 4054 are illustrated. In operation, the inflation device 4056 is deflated as the abrading head assembly 4028 is positioned within the nucleus pulposus. In the illustrated embodiment, the diameter of the deflated inflation device 4056 is approximately five mm. In other embodiments, the diameter of the deflated inflation device 4056 can be more or less than approximately five mm. Once the abrading head assembly 4028 achieves the desired position within the nucleus pulposus, the inflated device 4056 can be inflated. In the illustrated embodiment, the diameter of the inflated inflation device 4056 is approximately fourteen mm. In other embodiments, the diameter of the inflated inflation device 4056 can be more or less than approximately fourteen mm.

The inflation device 4056 is provided with an inflating fluid through conductors (not shown) positioned within a service cable 4058. The inflating fluid can be the same as the inflating fluid provided for the inflating device 2708 illustrated in FIGS. 22a and 23a. Once the inflation device 4056 is inflated, the inflation device 4056 is configured to maintain the abrading head assembly 4028 in a desired diameter thereby advantageously preventing potential damage to the adjacent vertebral plates.

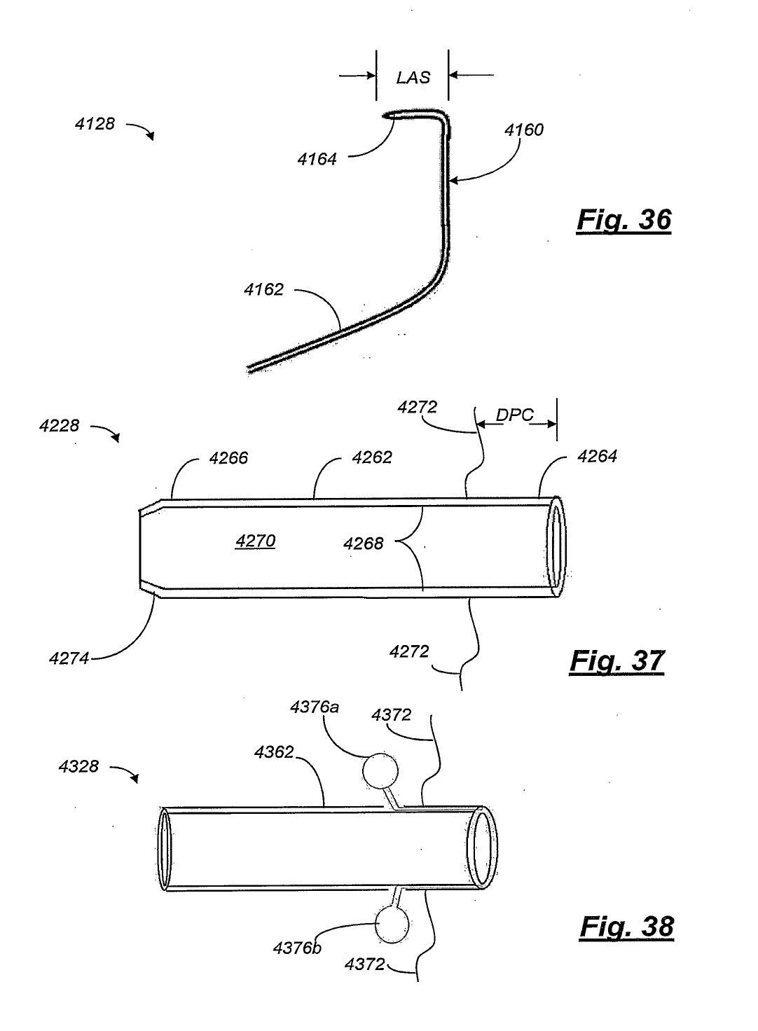

Referring now to FIG. 36, another embodiment of an ablating head assembly 4128 is illustrated. The ablating head assembly 4128 includes a wand 4160 that is connected to a cable 4162. An ablating surface 4164 is positioned at one end of the wand 4160. In the illustrated embodiment, the ablating surface 4164 is a RF-based ablating system. In other embodiments, the ablating surface 4164 can include other ablating systems. The ablating surface 4164 has a length LAS. In the illustrated embodiment, the length LAS of the ablating surface 4164 is in a range of from about five mm to about ten mm. Alternatively, the length LAS of the ablating surface 4164 can be less than about five mm or more than about ten mm. In operation, the ablating head assembly 4128 is positioned within the nucleus pulposus without the use of a guide wire. However, the ablating head assembly 4128 can be used with a guide wire (not shown) if desired. Once in position, the ablating head assembly 4128 ablates the nucleus pulposus through circumferential or back and forth motion of the RF-based ablating surface 4164. The RF waves can operate within any desired frequency. The cable 4162 provides the ablating surface 4164 with the RF waves in any desired manner. If desired, a contralateral pedicle may also be utilized for an additional discectomy or for providing access to the disc space for irrigation or suction during the discectomy procedure. Alternatively, the service cable 4162 can be inserted into the disc space unilaterally and may also house irrigation and suction lines (not shown) in addition to the discectomy device.

After the insertion device has been urged through the pedicle and into the vertebra, and after the curved bone tunnel has been created, the surgical team can access the nucleus pulposus and the resulting area between the adjacent vertebrae for the surgical procedure. Access to the opening within the pedicle and the curved bone tunnel can be maintained by use of a portal cannula, such as indicated generally at 4228 in FIG. 37. In this first embodiment, the portal cannula 4228 includes an elongated center portion 4262, a distal end 4264, and a proximal end 4266. The center portion 4262 includes an inner wall surface 4268 that is configured to form a portal cannula passage 4270. The portal cannula passage 4270 extends from the distal end 4264 to the proximal end 4266 of the portal cannula 4228.

In operation, the portal cannula 4228 is inserted through skin 4272 of a patient such that the proximal end 4266 of the portal cannula 4228 is aligned with the opening (not shown) formed in the pedicle (not shown) by the insertion device (not shown), as described above. The proximal end 4266 of the portal cannula 4228 can be anchored to the pedicle. In one embodiment, the proximal end 4266 can be anchored to the pedicle by pins (not shown). However, it should be understood that the proximal end 4266 of the portal cannula 4228 can be anchored to the pedicle in any desired manner. Once the portal cannula 4228 is anchored to the pedicle, the portal cannula 4228 extends outward from the skin 4272 a distance DPC. In the illustrated embodiment, the distance DPC is in a range of from about eight mm to about ten mm. In other embodiments, the distance DPC can be less than about eight mm or more than about ten mm. The portal cannula passage 4270 is used as desired for access to the nucleus pulposus and the resulting area between the adjacent vertebrae.

As shown in FIG. 37, the proximal end 4266 of the portal cannula 4228 can include an optional tapered region 4274. The tapered region 4274 is adapted to direct surgical implements passing through the portal cannula passage 4270 into the pedicle opening. However, the tapered region 4274 is optional and the portal cannula 4228 can be practiced without the tapered region 4274.

Referring now to FIG. 38, a second embodiment of a portal cannula, indicated generally at 4328, is illustrated. In this embodiment, the portal cannula 4328 is substantially the same as the portal cannula 4228 illustrated in FIG. 37, with the exception that the portal cannula 4328 is anchored in position by one or more inflatable devices. Only two of such inflatable devices 4376a and 4376b are illustrated in FIG. 38. However, more or less than two of such inflatable devices can be used. The inflatable devices 4376a and 4376b are configured to be deployed in a deflated condition in soft tissue under the skin 4372. Once positioned, the inflatable devices 4376a and 4376b can be inflated, thereby anchoring the portal cannula 4328 in a desired position. In the illustrated embodiment, the inflatable devices 4376a and 4376b have a balloon shape. Alternatively, the inflatable devices 4376a and 4376b can have any desired shape. The inflatable devices 4376a and 4376b may be inflated using a saline solution or any other desired liquid or fluid, including air.

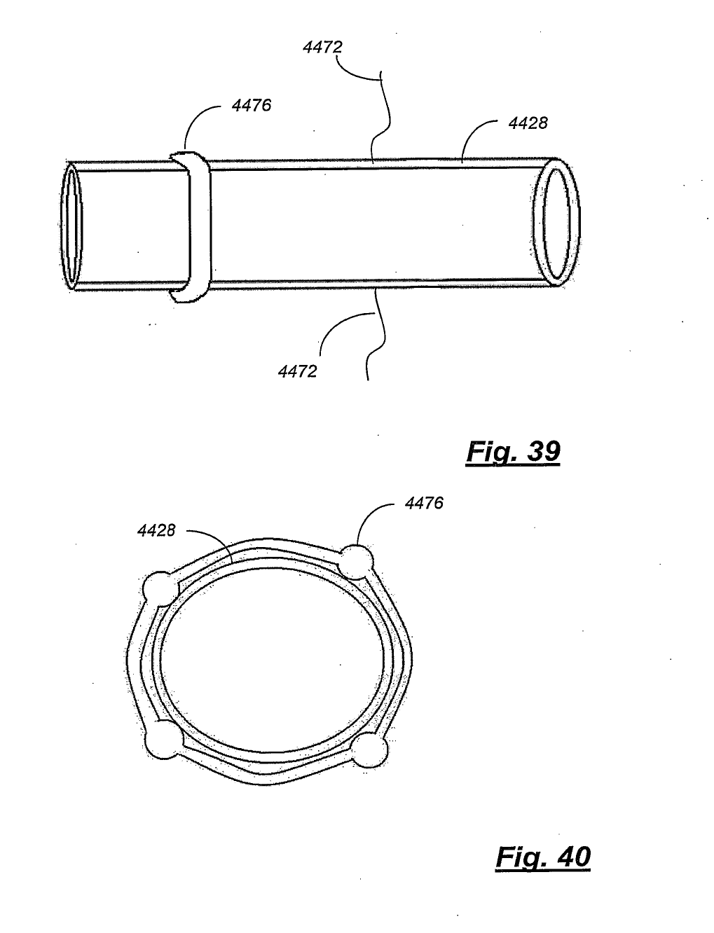

A third embodiment of an inflatable device, indicated generally at 4476, for anchoring a portal cannula 4428 is illustrated in FIGS. 39 and 40. In this third embodiment, the inflatable device 4476 is an inflatable collar. As discussed above, the inflatable collar 4476 is initially provided in a deflated condition in soft tissue under the skin 4472. Once positioned, the inflatable collar 4476 is inflated, thereby anchoring the portal cannula 4428 in a desired position. The inflatable device 4476 can have any desired configuration including but not limited to, linear, spiral, or random orientations. It should also be appreciated that the inflatable device 4476 can have any desired shape. While the embodiment shown in FIGS. 39 and 40 illustrates a single inflatable collar 4476, it should be understood that more than one inflatable collar 4476 can be used.

It should be understood that the securing of the portal cannula 4428 in a desired location with the help of inflatable devices can also be used for other surgical procedures, including non-limiting examples such as arthroscopic surgery and other minimal access orthopedic or non-orthopedic surgical procedures. The inflatable collar 4476 may be particularly useful during arthroscopic surgery, as it may minimize the extravasation of the irrigation fluid in the extra-articular soft tissues and therefore can substantially prevent the occurrence of compartment syndromes.

The progress of discectomy may be monitored with the help of a flexible endoscopic camera that can allow visualization of the disc space. The endoscopic camera can be integrated into the service cable utilizing either RF or mechanical disc ablation devices. The endoscopic camera can also be used as a separate unit to visualize the interior of the disc space through the bone tunnel. Alternatively, a discogram may be obtained prior to performing discectomy. In this method, a radio-opaque dye is injected into the disc space through the curved bone tunnel after the guide wire has been advanced into the desired location within the intervertebral disc. The clearance of the dye on the fluoroscopic images as the discectomy is carried out defines the extent of discectomy performed.

Once the discectomy and vertebral end plate preparation is completed, fusion material (not shown) can be inserted into the disc space through the curved bone tunnel in the vertebra. In one embodiment, the fusion material can be tamped in to the disc space with flexible polymeric or shape memory material. Alternatively, the fusion material can be positioned in the disc space with other desired surgical techniques. Optionally, the fusion material can be back-filled into a portion of the curved bone tunnel to decrease the risk of subsidence and bone graft migration. Subsequently, the fused vertebral assembly can be completed by placement of vertebral support devices, such as for example pedicle anchors and rods.

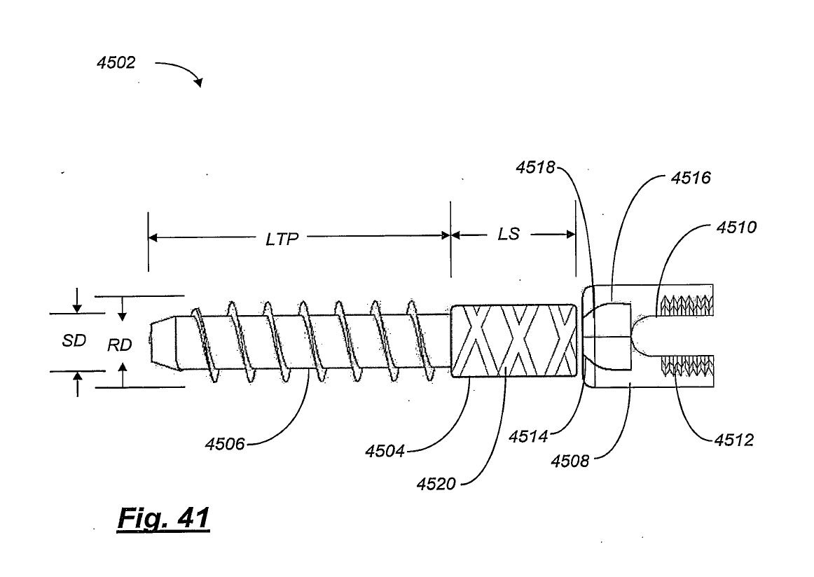

FIG. 41 illustrates a pedicle anchor, indicated generally at 4502, that can be used in the transpedicular method. The pedicle anchor 4502 is configured to fix desired medical implements to vertebral pedicles. In the illustrated embodiment, the pedicle anchor 4502 includes a proximal shank portion 4504 with an outer surface that is compatible with minor violations of adjacent vertebral elements. The minor violations of the adjacent vertebral elements may cause nerve root irritation. In one embodiment, the proximal shank portion 4504 can have a substantially smooth outer surface. Generally, the smooth outer surface of the proximal shank portion 4504 is configured to minimize nerve root damage in the event that a minor violation of the pedicle cortex. In other embodiments, the proximal shank portion 4504 can have other outer surface finishes or coatings configured to be compatible with minor violations of adjacent vertebral elements. The proximal shank 4504 may have small grooves or projections 4520 that are randomly, linearly, or spirally oriented. The projections or grooves 4520 can be configured to create a rough surface across the entire exterior surface of the proximal shank 4504 or only a portion of the exterior surface of the proximal shank 4504. The grooves or projections 4520 on the proximal shank 4504 can be located strategically away from the area in proximity of the neural elements. Optionally, the proximal shank portion 4504 may also be coated symmetrically or asymmetrically with hydroxyapatite or other desired agents for improved bonding with the native bone of the pedicle.

The pedicle anchor 4502 also includes a threaded portion 4506 and a head 4508. The threaded portion 4506 is configured for insertion into a vertebra. The threaded portion 4506 has a root diameter RD. In the illustrated embodiment, the root diameter RD is seven mm. In other embodiments, the root diameter RD can be more or less than seven mm. The threaded portion 4506 can have any desired thread diameter, thread pitch, and number of threads. The threaded portion 4506 has a length LTP. In the illustrated embodiment, the length LTP is approximately forty-five mm. In other embodiments the length LTP can be more or less than approximately forth-five mm. The length LTP of the threaded portion 4506 can extend any desired distance into a vertebra.

The shank portion 4504 is configured for positioning in the pedicle where a large part of the cancellous core is removed during formation of the bone tunnel. The external cortical bone can inadvertently become too thin and a threaded conventional pedicle screw may cause irritation of the adjacent nerve root by breaching the cortex. The shank portion 4504 can have any desired shank diameter SD, but generally the diameter of the proximal shank portion 4504 is equal to or larger than the root diameter RD of the threaded portion 4506. A larger cross section of the proximal shank portion 4504 may also “fill” the inside of the pedicle more completely, thereby providing improved stability. The shank portion 4504 has a length LS. In the illustrated embodiment, the length LS of the shank portion 4504 is approximately fifteen mm and is configured to be placed within the pedicle, but it can extend any desired distance into a vertebra. Alternatively, the shank portion 4504 can have any desired length LS.

The head 4508 includes a yoke 4510 and a threaded portion 4512. The yoke 4510 is configured to receive a medical implement (not shown). In the illustrated embodiment, the yoke 4510 is generally U-shaped. However, the yoke 4510 can have any other desired shape that is adapted to receive a medical implement. The head 4508 includes a threaded portion 4512 and can be configured to correspond with a threaded portion of a corresponding retainer (not shown). The threaded portion 4512 of the head 4508 and the corresponding threaded portion of the retainer can have any desired thread characteristics sufficient to retain a desired medical implement in the yoke 4510 and prevent the medical implement from moving in a direction relative to the yoke 4510. The retainer can be rotated in any desired manner to engage the threaded portion 4512 of the head 4508. While the embodiment shown in FIG. 41 includes a threaded portion 4512 corresponding to a threaded retainer, it should be understood that a medical implement could be retained in the yoke 4510 with other desired structures, mechanisms and devices.

The head 4508 is pivotally attached to the shank portion 4504 at a pivot point 4514. The pivot point 4514 is configured to permit the head 4508 to pivot in any desired direction to align with a medical implement. In the illustrated embodiment, the pivot point 4514 includes a ball 4516 and socket 4518. Alternatively, the pivot point 4514 can be any desired structure, mechanism, or device sufficient to permit the head 4508 to pivot in a desired direction to align with a medical implement. In the illustrated embodiment, the pedicle anchor 4502 is made from titanium. In other embodiments, the pedicle anchor 4502 can be made from other suitable bio-compatible materials. The pedicle anchor 4502 can have any desired surface, finish or coating. In anther embodiment, a collar (not shown) having a smoother outer surface may be placed in the pedicle opening though which a conventional pedicle anchor may be inserted. In one embodiment, the collar can be made of a bio-compatible material such as titanium. In other embodiments, the collar can be made of other bio-compatible materials such as for example polyetheretherketone (“PEEK”), bone dowel, or bioresorbable materials.

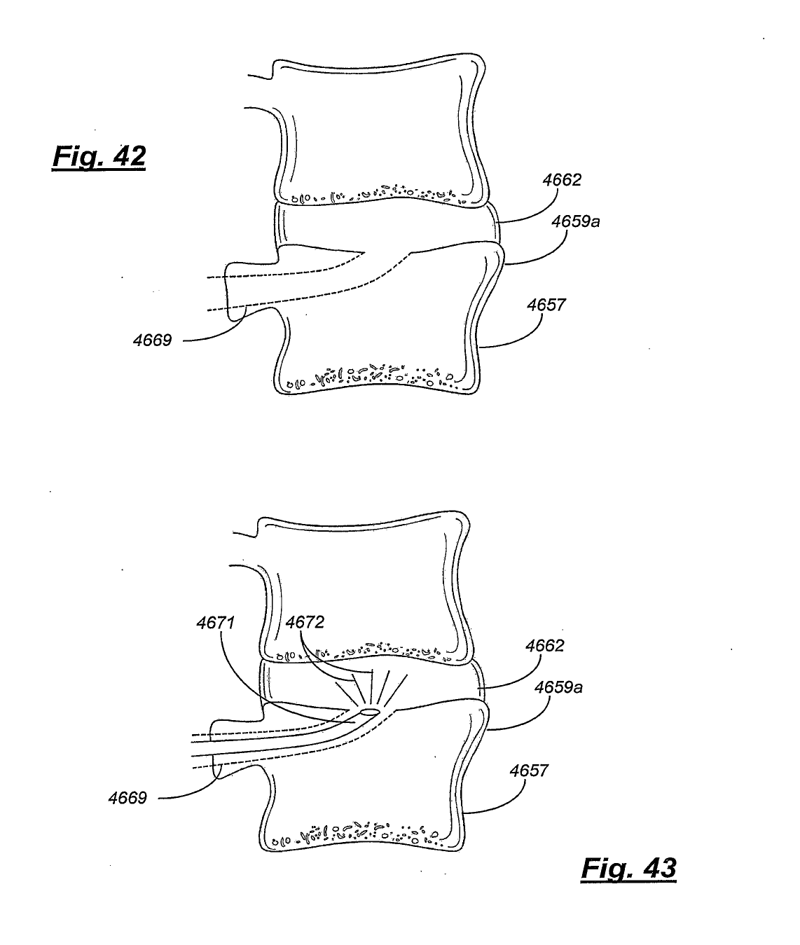

Referring now to FIGS. 42-44, other embodiments of the invention are illustrated. In these embodiments, other modalities, such as high frequency ultrasound probes, can be utilized for vertebral end plate preparation. As shown in FIG. 42, a bone tunnel 4669 is created in a vertebra 4657 in such a manner that it exits an end plate 4659a of the vertebra 4657 as close to the center of a disc 4662 as possible, as determined by anteroposterior and lateral radiographs. Next, as shown in FIG. 43, an ultrasound probe 4671 is positioned within the bone tunnel 4669 and adjacent the disc 4662. In the illustrated embodiment, the ultrasound probe 4671 provides high frequency ultrasound beams 4672 that are focused on the nucleus pulposus of the disc 4662. The high frequency ultrasound beams 4672 generate thermal energy in a range of from about 60° F. to about 80° F. in the desired location resulting in coagulation necrosis of the nucleus pulposus. The focused ultrasound beam substantially minimizes the scatter and prevents unintentional coagulation of surrounding structures. In other embodiments, the high frequency ultrasound beams can emanate in a symmetric reverse conical manner.

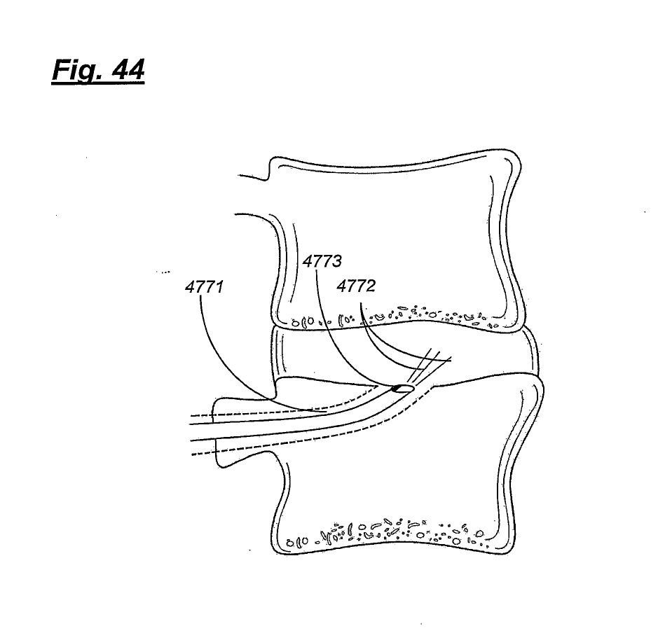

Referring now to FIG. 44, an ultrasonic probe 4771 can produce preferential directional ultrasonic beams 4772. In the illustrated embodiment, the directional beams 4772 are formed by use of a probe hood 4773 formed at the tip of the ultrasound probe 4771. The probe hood 4773 can be any desired structure, mechanism, or device that is configured to direct ultrasound beams in a preferential direction. In the illustrated embodiment, the ultrasonic probe 4771 having the probe hood 4773 is configured for rotation, thereby producing coagulation of the nucleus pulposus with substantial control. In other embodiments, the ultrasonic probe 4771 having the probe hood 4773 can be oriented to prevent rotation. In still other embodiments, the ultrasonic probe 4771 can be utilized over a guide wire (not shown) positioned within the disc space.

In other to mitigate thermal damage to the annulus fibrosus, a cooling catheter (not shown) can be positioned along the peripheral of the disc space adjacent to the interior of the annulus fibrosus. The catheter can have any desired structure, including the non-limiting examples of a radial or spiral structure. The cooling catheter can have several perforations for slow and controlled egress of a coolant, such as normal saline or other desired bio-compatible fluids. In certain embodiments, the cooling catheter can be inserted from the contralateral pedicle. Alternatively, the cooling catheter can be inserted from a traditional transannular approach, such as for example that approach used for discography. The cooling catheter can be made of a suitable bio-compatible material and can be insulated for protection against substantial thermal damage.

The above detailed description of this invention is given for explanatory purposes. It will be apparent to those skilled in the art that numerous changes and modifications other than those cited can be made without departing from the scope of the invention. Accordingly, the whole of the foregoing description is to be construed in an illustrative and not a limitative sense, the scope of the invention being defined by the appended claims.

What is claimed is:

1. A probe assembly configured for ablation of disc material, the probe assembly comprising;
a plurality of probe filaments;
an inflatable device positioned adjacent to a portion of the probe filaments; and
a cable connected to the probe filaments and the inflatable device;
wherein the probe filaments are configured for ablation of disc material using RF radiofrequency waves.

2. The probe assembly of claim 1 wherein the inflatable device is configured to be deployed in a deflated condition and can be inflated after deployment in a disc area.

3. The probe assembly of claim 1 wherein the probe filaments are guided into and around the disc area by a guide wire.

4. The probe assembly of claim 1 wherein a guide support is attached to the probe filaments, the guide support being configured for supporting the guide wire.

5. The probe assembly of claim 4 wherein the guide support includes cutouts.

6. The probe assembly of claim 3 wherein the probe filaments include an external passage configured for the guide wire.

7. The probe assembly of claim 1 wherein the probe filaments are configured for passage through a curved bone tunnel to the disc area.

8. The probe assembly of claim 1 wherein the probe filaments are configured to produce directional RF radiofrequency waves.

9. An abrading assembly configured to abrade disc material, the abrading assembly comprising:
an abrading head;
an actuator connected to the abrading head, the actuator configured to rotate the abrading head;
wherein the abrading head and the actuator are configured to be guided by a guide wire.

10. The abrading assembly of claim 9 wherein a portion of the abrading head is covered by a hood.

11. The abrading assembly of claim 9 wherein a guide support is connected to the actuator and configured to support the guide wire.

12. The abrading assembly of claim 9 wherein an inflatable device is positioned within the abrading head, and wherein the inflatable device is configured to be deployed in a deflated condition and further configured to be inflated after deployment in a disc area, wherein the inflated device increases the diameter of the abrading head.

13. A probe assembly configured for ablation of disc material from between opposing vertebrae, the probe assembly comprising;
a probe element having a fixed diameter;
a guide wire connected to the probe element and configured to guide the probe element through a curved bone tunnel formed in one vertebra;
a cable connected to the probe element;
wherein the probe element is configured for ablation of disc material using RF radiofrequency waves.

14. The probe assembly of claim 30 wherein the guide wire passes through an external cannulated guide support connected to the probe element.
